(19) United States
(12) Patent Application Publication
(10) Pub. No.: US 2014/0112953 A1
(43) Pub. Date: Apr. 24, 2014

(54) INACTIVATED DENGUE VIRUS VACCINE

(71) Applicants: Veronique Henderickx, Rixensart (BE); Olivier Le Bussy, Rixensart (BE); Dominique Ingrid Lemoine, Rixensart (BE); Frederic Mathot, Rixensart (BE)

(72) Inventors: Veronique Henderickx, Rixensart (BE); Olivier Le Bussy, Rixensart (BE); Dominique Ingrid Lemoine, Rixensart (BE); Frederic Mathot, Rixensart (BE)

(73) Assignee: Glaxo SmithKline Biologicals sa, Rixensart (BE)

(21) Appl. No.: 14/119,902

(22) PCT Filed: May 25, 2012
(86) PCT No.: PCT/EP2012/059879

§ 371 Date: Nov. 25, 2013

Related U.S. Application Data
(60) Provisional application No. 61/570,966, filed on Dec. 15, 2011.
(60) Provisional application No. 61/490,205, filed on May 26, 2011.

(51) Int. Cl. A61K 39/12

(52) U.S. Cl. CPC A61K 39/12
USPC 424/218.1

(57) ABSTRACT

The present invention provides formulations of an immunogenic composition containing a purified inactivated Dengue virus, and method for producing them.

### CROSS-REFERENCE TO RELATED APPLICATIONS

This application claims benefit of the earlier filing dates of U.S. Provisional Applications 61/490,205, filed 26 May 2011 and 61/570,966, filed 15 Dec. 2011, the disclosures of which are incorporated herein.

### COPYRIGHT NOTIFICATION PURSUANT TO 37 C.F.R. §1.71(E)

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND

Dengue is an acute viral disease of man which is transmitted by mosquitos. It is endemic in the tropics and subtropics, worldwide, where an estimated 100,000,000 cases occur annually. Although relatively rare, Dengue hemorrhagic fever (DHF) and Dengue shock syndrome (DSS) are significant causes of death in children. At present, there is no vaccine to protect against Dengue and attempts to prevent disease by controlling the mosquito vector have proven largely ineffective. Thus, there remains a need for a safe and effective vaccine to protect against disease caused by Dengue virus.

### BRIEF SUMMARY

The present invention disclosure concerns the formulation of compositions that elicit an immune response against Dengue virus.

### BRIEF DESCRIPTION OF THE DRAWINGS

FIG. 1 is a schematic illustration of the generic formula of a poloxamer surfactant: α-Hydro-ω-hydroxypoly(oxyethylene)poly(oxypropylene) poly(oxyethylene)triblock copolymer.

FIGS. 2A and B are flow charts that illustrate exemplary processes for purification and inactivation of an immunogenic composition comprising purified inactivated Dengue virus. FIGS. 2C and D are flow charts that illustrate an alternative process for purification and inactivation.

FIGS. 3A and B are flow charts that illustrate exemplary processes for formulation of an immunogenic composition comprising purified inactivated Dengue virus.

FIGS. 4A-B are tables illustrating representative results of product characterization following formulation of immunogenic compositions comprising purified inactivated Dengue virus.

FIGS. 5A-C are tables illustrating representative results of product characterization following lyophilization and reconstitution.

FIGS. 6A and B are graphical representations of stability characteristics (A: Intrinsic Fluorescence 280/320; B: ELISA).

### DETAILED DESCRIPTION

### Introduction

This disclosure concerns the formulation of immunogenic compositions. In particular, this disclosure relates to formulations of compositions, such as bulk vaccine preparations and immunogenic compositions, containing one or more strains of purified inactivated Dengue virus. The formulations disclosed herein increase recovery and stability of immunogenic compositions containing purified inactivated Dengue viruses, facilitating their production, storage and distribution.

A first aspect of this disclosure relates to compositions that include one or more purified inactivated Dengue viruses, in combination with a buffering agent and a surfactant. Favorably, such compositions are bulk preparations of inactivated Dengue virus suitable for formulation into immunogenic compositions (e.g., vaccines to prevent infection by and/or disease due to Dengue virus). Addition of a selected surfactantenhances recovery of an antigenically preserved inactivated Dengue virus, e.g., as compared to formulations that do not include a surfactant. Formulations of purified inactivated Dengue virus containing a surfactant possess the favorable characteristic of reducing nonspecific adsorption and/or aggregation of the inactivated virus, e.g., during lyophilization, storage and reconstitution.

The compositions disclosed herein can include one or more than one serotype of Dengue virus. Commonly, the compositions include a plurality of Dengue viruses from more than one serotype, that is Dengue serotype 1, Dengue serotype 2, Dengue serotype 3 and/or Dengue serotype 4 (DEN-1, DEN-2, DEN-3, and/or DEN-4, respectively). For example, the composition can include two, three or four different viruses from different serotypes of Dengue virus. In a specific example, the composition includes four different purified inactivated Dengue viruses, each of a different serotype (or capable of eliciting an immune response specific for each of the different serotypes of Dengue virus. Thus, the composition favorably includes four different purified inactivated Dengue viruses that elicit an immune response to all of DEN-1, DEN-2, DEN-3 and DEN-4. The virus(es) can be selected from among wild-type viruses (i.e., propagated from or corresponding to virulent virus from a naturally occurring isolate), or the virus(es) can be selected from attenuated viruses. A selected virus can be a recombinant virus. For example, a recombinant virus can be a chimeric virus, e.g., a virus having a nucleic acid from a Dengue virus and a nucleic acid from another flavivirus, such as a different Dengue virus, a Yellow Fever virus, or a Japanese Encephalitis virus. Typically, a chimeric virus includes one or both of a Dengue M and a Dengue E protein. A single composition can include one or more wild-type virus, one or more attenuated virus, one or more recombinant virus, and/or one or more chimeric virus, in any combination.

The purified inactivated Dengue virus can be inactivated using chemical, physical and/or irradiating inactivating agents, alone or in any combination. The purified inactivated Dengue virus can be inactivated by exposure to formaldehyde, betapropiolactone (BPL), hydrogen peroxide, ultraviolet irradiation and gamma irradiation, or combination of any of these techniques.

Typically, a single human dose of the immunogenic composition contains at least 0.1 μg, 0.2 μg, at least 0.25 μg, at least 0.3 μg, at least 0.33 μg, at least 0.4 μg, at least 0.5 μg, at least 1.0 μg, or at least 2.0 μg, or at least 3.0 μg, or at least 5.0 μg, or at least 10.0 μg, (or any amount between 0.1 and 10.0 μg) of each serotype of virus. Typically, a single human dose of the immunogenic composition contains no more than 100 μg of each serotype of virus, for example, no more than 90 μg, or no more than 80 μg, or no more than 75 μg, or no more than 70 μg, or no more than 60 μg, or no more than 50 μg, or no more than 40 μg, or no more than 30 μg, or no more than 20 μg, or no more than 10 μg (or any amount between 10 and 100 μg) of each serotype of virus. For example, a single human dose of the immunogenic composition can include between 0.1 and 10 μg, or between 0.25 and 5 μg, e.g., administered in a volume of between 0.05 and 2 ml, such as in a volume 0.5 and 1.5 ml.

In certain embodiments, the purified inactivated Dengue virus(es) are adsorbed onto an aluminum salt (“alum”), such as aluminum hydroxide, aluminum phosphate or aluminum hydroxyphosphate. Where a plurality of Dengue viruses is included, each can be adsorbed onto the same aluminum salt, or different viruses can be adsorbed onto different aluminum salts. Thus, in one aspect, the present disclosure concerns an immunogenic composition that contains at least one purified inactivated Dengue virus adsorbed (e.g., preadsorbed) onto an aluminum salt, in combination with a buffer and a surfactant.

In the context of the immunogenic compositions disclosed herein (and the bulk preparations from which the finished immunogenic compositions are formulated), the surfactant is selected to be suitable for administration to a subject, particularly a human subject. In certain embodiments, the surfactant is selected to be suitable for parenteral administration, e.g., for intramuscular, subcutaneous, transcutaneous or intradermal administration.

Exemplary surfactants suitable for the Dengue compositions disclosed herein include poloxamer surfactants, as well as other surfactants suitable for administration to a human subject. Thus, a suitable surfactants (in addition to poloxamer surfactants) can be selected from the group consisting of: polysorbate surfactants, octoxinol surfactants, polidocanol surfactants, polyoxyl stearate surfactants, polyoxyl castor oil surfactants, N-octyl-glucoside surfactants, macrogol 15 hydroxy stearate, and combinations thereof. In certain embodiments, Poloxamer surfactants are particularly suitable for formulations in which the purified inactivated Dengue virus(es) are not adsorbed onto an aluminum salt.

Poloxamer surfactants are polyethylene-polypropylene glycol linear copolymers. Commercially, these are often referred to as Pluronic surfactants. In certain embodiments, the poloxamer surfactant is selected from a polyethylene-polypropylene glycol copolymer with an average molecular weight of at least about 1000 kD, and an average molecular weight of no more than about 15,000 kD. In one specific embodiment, the immunogenic composition is formulated with a polyethylene-polypropylene glycol copolymer, poloxamer 188, which is sold commercially under the trademarks Pluronic™ F 68, Lutrol™ F 68, and Kolliphor™ P188, which has an average molecular weight of 8600 kD, with a polyoxypropylene molecular weight of 1800 g/mole and an 80% polyoxyethylene content.

The compositions (bulk preparations and immunogenic compositions) also include one or more buffering agents. Dengue virus loses immunogenicity under acid conditions, thus the buffering agent is selected to maintain the pH near or above neutral. The buffering agent or agents, is typically selected to maintain the pH of the composition at or above pH 6.4, preferably above pH 6.8, and most preferably above pH 7.0, e.g., at or about pH7.4. The buffering agent is selected to maintain the desired pH in the context of the other components of the formulated immunogenic composition, taking into consideration that certain additional components (e.g., certain adjuvants) may require adjusting the quantity or choice of buffering agent. In one embodiment, the buffering agent includes one or both of sodium phosphate and potassium phosphate. In another embodiment, the buffering agent includes Tris(hydroxymethyl)aminomethane. (“Tris”).

The bulk preparations and immunogenic compositions can also include additional components, such as one or more mineral salts, e.g., to modify or maintain tonicity in a desired range. Most commonly, the salt is a mineral salt, such as sodium chloride. Such a salt is favorably added in the amount necessary to maintain the formulated composition at or near isotonic. The precise amount differs depending on the other components in the formulation, most particularly on the choice of buffering agent(s), and can be determined without undue experimentation by those of ordinary skill in the art.

The bulk preparations and immunogenic compositions disclosed herein can also include one or more excipient to enhance structural and/or immunological stability (or to modify other properties of the formulation, such as tonicity) of the purified inactivated Dengue virus in solution and/or during processing, e.g., lyophilization. In some embodiments, the excipient includes a glass forming sugar or polyol. In certain embodiments, the glass forming sugar or polyol is selected from the group consisting of: sucrose, trehalose, mannose, mannitol, raffinose, lactitol, sorbitol and lactobionic acid, glucose, maltulose, iso-maltulose, lactulose, maltose, lactose, iso-maltose, maltitol, palatinit, stachyose, melezitose, dextran or a combination thereof. In one specific embodiment, the excipient comprises sucrose. Optionally, the sugar or polyol can be used in combination with an amino acid, such as glycine, alanine, arginine, lysine and/or glutamine.

In certain embodiments, the composition is a liquid formulation, e.g., a solution or suspension. In other embodiments, the composition is prepared lyophilized, and resuspended prior to administration. For example, the immunogenic composition can be formulated in an isotonic liquid formulation for administration by injection.

In certain embodiments, the immunogenic composition is formulated for administration to a human subject. For administration to a human subject, the immunogenic composition can be formulated in a single dose amount of at least 0.05 ml and no more than 2 ml, such as a single dose amount of between 0.5 and 1.5 mls.

Optionally, the immunogenic compositions disclosed herein can include an adjuvant. In some embodiments, e.g., embodiments in which the purified inactivated Dengue virus is adsorbed onto alum, the aluminum salt serves as an adjuvant. In other embodiments, the adjuvant is an aluminum-free adjuvant. Whether combined with, e.g., adsorbed onto, alum or not, the adjuvant can include one or more immunostimulatory components. The immunostimulatory component can include one or more of: an oil and water emulsion, a liposome, a lipopolysaccharide, a saponin, and an oligonucleotide, as described in more detail hereinbelow.

Another aspect of this disclosure relates to methods for formulating bulk antigen preparations and immunogenic composition comprising one or more purified inactivated Dengue viruses. Such a method involves: providing a solution comprising a buffering agent and a surfactant; and admixing with the solution one or more purified inactivated Dengue viruses. In some embodiments, the one or more purified inactivated Dengue viruses are adsorbed onto an aluminum salt (e.g., to produce a pre-adsorbed bulk preparation of inactivated Dengue virus) prior to admixing with the solution. Typically, a single strain of purified inactivated Dengue virus is adsorbed onto the aluminum salt (e.g., aluminum hydroxide, aluminum phosphate or aluminum hydroxyphosphate) to produce a pre-adsorbed monobulk. To produce a multivalent immunogenic composition, the individual monobulks are then combined in the desired ratio (e.g., 1:1:1:1 based on weight, or adjusted based on relative immunogenicity) with the solution containing the buffering agent and the surfactant.

Typically, the purified inactivated Dengue virus(es) are added to a solution suitable (in final formulation) for parenteral administration. In some embodiments, the solution is an isotonic solution. In some embodiments, the solution also includes one or more excipient, such as a salt and/or a glass forming sugar or polyol.

In an embodiment, to water for injection (e.g., sterile, endotoxin-free water), a glass forming sugar or polyol, a buffering agent, a salt and surfactant (as discussed above) are added, for example, in sequential order. The purified inactivated Dengue virus(es) as discussed above is/added to the prepared solution.

In some embodiments, the method then involves lyophilizing the solution (e.g., the bulk preparation) containing the purified inactivated Dengue virus(es) to produce a lyophilized composition. In embodiments involving the lyophilization of the immunogenic composition, e.g., for storage and/or distribution, the lyophilized composition is typically resuspended in a suitable amount, e.g., 0.05-2 mls, typically between 0.5 and 1.5 mls, for example, 0.5 or 1.0 or 1.5 mls, of a pharmaceutically acceptable solution, such as water for injection, prior to administration. Optionally, the pharmaceutically acceptable solution includes at least one immunostimulatory component, as disclosed above.

In another aspect, this disclosure concerns methods for reducing nonspecific adsorption and/or aggregation of a purified inactivated Dengue virus (or plurality thereof), or composition containing the same, by formulating the inactivated Dengue virus(es) as described above.

In yet another aspect, this disclosure relates to a method for enhancing recovery of an antigenically preserved inactivated Dengue virus (or plurality thereof), or composition containing the same, by formulating the inactivated Dengue virus(es) as described above.

### TERMS

Unless otherwise explained, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 20 μg, it is intended that the concentration be understood to be at least approximately (or “about” or “˜”) 20 μg.

Although methods and materials similar or equivalent to those described herein can be used in the practice or testing of this disclosure, suitable methods and materials are described below. The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof. The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of terms are provided. Additional terms and explanations can be provided in the context of this disclosure.

A “bulk preparation” of an inactivated Dengue virus is used herein to refer to a Dengue virus in the final antigenic form, with respect to purification and inactivation, intended for administration to a subject. A bulk preparation or bulk formulation can be further processed, e.g., by dilution, concentration, such as by lyophilization and resuspension, and/or packaged, e.g., into multidose or single dose vials or syringes for administration as an immunogenic composition or vaccine.

The term “purification” (e.g., with respect to a pathogen or a composition containing a pathogen, such as a Dengue virus) refers to the process of removing components from a composition, the presence of which is not desired. Purification is a relative term, and does not require that all traces of the undesirable component be removed from the composition. In the context of vaccine production, purification includes such processes as centrifugation, dialization, ion-exchange chromatography, and size-exclusion chromatography, affinity-purification or precipitation. Thus, the term “purified” does not require absolute purity; rather, it is intended as a relative term. Thus, for example, a purified virus preparation is one in which the virus is more enriched than it is in its generative environment, for instance within a cell or population of cells in which it is replicated naturally or in an artificial environment. A preparation of substantially pure viruses can be purified such that the desired virus or viral component represents at least 50% of the total protein content of the preparation. In certain embodiments, a substantially pure virus will represent at least 60% or at least 70%, such as at least 80%, at least 85%, at least 90%, or at least 95% or more of the total protein content of the preparation. Alternatively, the purification of a virus preparation can be assessed as the reduction in contaminants, such as host cell proteins, in the preparation. Accordingly, a preparation of substantially pure virus (e.g., purified inactivated Dengue virus) typically includes less than 30% or less than 25% residual host cell proteins. For example, a bulk preparation or immunogenic composition comprising a purified inactivated Dengue virus can include less than 20% residual host cell protein, or even less than 15% or 10% or less (e.g., measured on a wt/wt basis).

The term “inactivated” in the context of a Dengue virus vaccine means that the antigenic component (e.g., virus) is incapable of replication in vivo or in vitro. For example, the term inactivated encompasses a virus that has been replicated, e.g., in vitro, and then killed using chemical or physical means such that it is no longer capable of replicating. The term can also include antigens produced by further processing (e.g., splitting, fractionation, and the like), and components produced by recombinant means, e.g., in cell culture.

An “adjuvant” is an agent that enhances the production of an antigen-specific immune response as compared to administration of the antigen in the absence of the agent. Common adjuvants include aluminum containing adjuvants that include a suspensions of minerals (or mineral salts, such as aluminum hydroxide, aluminum phosphate, aluminum hydroxyphosphate) onto which antigen is adsorbed. Other adjuvants include one or more immunostimulatory component that contributes to the production of an enhanced antigen-specific immune response. Immunostimulatory components include oil and water emulsions, such as water-in-oil, and oil-in-water (and variants thereof, including double emulsions and reversible emulsions), liposaccharides, lipopolysaccharides, immunostimulatory nucleic acids (such as CpG oligonucleotides), liposomes, Toll-like Receptor agonists (particularly, TLR2, TLR4, TLR7/8 and TLR9 agonists), and various combinations of such components. Adjuvants can include combinations of immunostimulatory components.

An “immunogenic composition” is a composition of matter suitable for administration to a human or animal subject (e.g., in an experimental setting) that is capable of eliciting a specific immune response, e.g., against a pathogen, such as Dengue virus. As such, an immunogenic composition includes one or more antigens (for example, whole purified virus or antigenic subunits, e.g., polypeptides, thereof) or antigenic epitopes. An immunogenic composition can also include one or more additional components capable of eliciting or enhancing an immune response, such as an excipient, carrier, and/or adjuvant. In certain instances, immunogenic compositions are administered to elicit an immune response that protects the subject against symptoms or conditions induced by a pathogen. In some cases, symptoms or disease caused by a pathogen is prevented (or treated, e.g., reduced or ameliorated) by inhibiting replication of the pathogen (e.g., Dengue virus) following exposure of the subject to the pathogen. In the context of this disclosure, the term immunogenic composition will be understood to encompass compositions that are intended for administration to a subject or population of subjects for the purpose of eliciting a protective or palliative immune response against Dengue (that is, vaccine compositions or vaccines).

An “immune response” is a response of a cell of the immune system, such as a B cell, T cell, or monocyte, to a stimulus. An immune response can be a B cell response, which results in the production of specific antibodies, such as antigen specific neutralizing antibodies. An immune response can also be a T cell response, such as a CD4+ response or a CD8+ response. In some cases, the response is specific for a particular antigen (that is, an “antigen-specific response”). If the antigen is derived from a pathogen, the antigen-specific response is a “pathogen-specific response.” A “protective immune response” is an immune response that inhibits a detrimental function or activity of a pathogen, reduces infection by a pathogen, or decreases symptoms (including death) that result from infection by the pathogen. A protective immune response can be measured, for example, by the inhibition of viral replication or plaque formation in a plaque reduction assay or ELISA-neutralization assay, or by measuring resistance to pathogen challenge in vivo.

A “subject” is a living multi-cellular vertebrate organism. In the context of this disclosure, the subject can be an experimental subject, such as a non-human animal, e.g., a mouse, a cotton rat, or a non-human primate. Alternatively, the subject can be a human subject.

A “buffering agent” is a compound or composition that alone or in combination increases the ability of a solution to maintain or resist change in pH when an acid or an alkali is added. The term buffering agent encompasses a wide variety of compounds and compositions, typically, either weak acids or weak bases, which when present in solution with their conjugate base or acid, respectively, can be used to maintain the pH at a desired value or within a desired range.

A “surfactant,” or surface active agent, is an amphiphilic molecules characterized by a hydrophilic head and a hydrophobic tail. When adsorbed at the surface of a liquid, a surfactant acts to lower the surface tension of the liquid, the interfacial tension between two liquids, or the tension between the liquid and a solid. A surfactant may act as detergent, wetting agent, emulsifier, foaming agent, and/or dispersant.

The compositions disclosed herein include one or more purified inactivated Dengue virus antigen. In various aspects, the compositions are manufactured bulk preparations of inactivated Dengue virus, e.g., in a liquid formulation, solid (e.g., lyophilized) preparations at a selected scale, or immunogenic compositions formulated for administration to a subject (typically a human subject). For example, the bulk preparations (whether liquid or solid) and/or immunogenic compositions can include a single strain of Dengue virus (i.e., a monovalent composition, such as a monovalent bulk preparation or monovalent immunogenic composition), or they can contain more than one strain of Dengue virus (i.e., a multivalent composition, such as a multivalent bulk preparation or multivalent immunogenic composition). Typically, a multivalent composition contains strains selected from different serotypes. Because there are four serotypes of Dengue virus which can cause disease, that is, Dengue type one (DEN-1), Dengue type two (DEN-2), Dengue type three (DEN-3) and Dengue type four (DEN-4), and because cross-reactive non-neutralizing antibodies are predisposing to more severe forms of Dengue disease, one representative of each serotype can be selected for inclusion into the bulk preparation and final vaccine in order to guarantee protection against disease from any of the four serotypes. Thus, in one embodiment, the immunogenic composition is a tetravalent composition that includes strains selected from each of the four serotypes of Dengue virus.

The viruses used as antigens can be selected from essentially any strain (or strains) of Dengue virus. For example, a virus strain can be selected for each serotype, which is chosen based on its conformity to a defined (e.g., consensus) sequence for the serotype, such as a DEN-1 consensus sequence, a DEN-2 consensus sequence, a DEN-3 consensus sequence, or a DEN-4 consensus sequence. Such a virus can be naturally occurring or synthetic. For example, a virus strain can be selected to correlate with a strain prevalent (e.g., a naturally occurring or “wild type” strain) in the area or population in which the vaccine is intended to be administered. Another option is to select strains for each serotype as a matter of convenience based on availability or prior experience. For example, exemplary strains are described in U.S. Pat. No. 6,254,873, which is incorporated by reference herein. Additional suitable strains are disclosed, e.g., in U.S. Pat. No. 7,226,602, which is also incorporated herein by reference. Additional strains can be found, for example, in the VBRC viral genome database (http://athena.bioc.uvic.ca/organisms/Flaviviridae/Dengue/Curated_genes), and the Dengue Virus Database (http://www.broad.mit.edu/annotation/viral/Dengue/ProjectInfo.html).

In the context of a purified inactivated Dengue virus vaccine, either virulent or attenuated strains can be used. Typically virulent strains propagate to higher titer in host cells, facilitating production at commercial scale. However, virulent strains require special care in handling to prevent infection of personnel involved in manufacturing. Attenuated strains, e.g., developed by adaptation to production in cultured cells and selection for reduced virulence and/or reduced replication in the mosquito vectors of Dengue, require fewer handling precautions but can be difficult to produce. Exemplary attenuated strains suitable for use in the context of an immunogenic composition containing an inactivated Dengue virus are described in WO 2000/057907 and U.S. Pat. No. 6,638,514, and WO 2000/058444 and U.S. Pat. No. 6,613,556, WO 2002/066621 (US Publication No. 2004052818), WO 2000/057904 (U.S. Pat. No. 6,528,065, WO 2000/057908, WO 2000/057909 (U.S. Pat. No. 6,511,667); WO 2000/057910 (U.S. Pat. No. 6,537,557), WO 2002/095075 (e.g., U.S. Pat. No. 7,226,602) and WO 2002/102828 (U.S. Pat. No. 7,569,383), which are incorporated herein by reference.

Chimeric “Dengue” viruses are also suitable in the context of the formulations disclosed herein. Such a chimeric virus typically expresses the Dengue virus envelope protein, for example, using a nucleic acid backbone of a different Dengue virus or of a different flavivirus, such as a Yellow Fever virus or a Japanese Encephalitis virus. Examples of chimeric Dengue viruses can be found in, e.g., WO 98/37911 (U.S. Pat. Nos. 6,696,281; 6,962,708), WO 96/40933 and WO 2001060847 (U.S. Pat. Nos. 7,094,411; 7,641,909; 8,025,887) and EP1159968 Methods for producing such chimeric Dengue virus can also be found in WO 03/101397. The disclosures of these published applications and patents are incorporated herein by reference for the purpose of providing exemplary chimeric Dengue viruses suitable for use in the context of the formulations and methods disclosed herein.

Thus, the strain(s) selected are typically chosen from among the numerous strains available to replicate in cells that are suitable for production of materials intended for human use (e.g., cells that are certified free of pathogens). For example, strains can be screened to identify those viruses that grow to the highest titers, for example from a titer of at least about 5×10⁶ pfu/ml, preferably at least 1×10⁷ pfu/ml or more in the cell line(s) of choice; (ii) selecting those strains of Dengue virus which grow to the highest titers in the cell line(s) of choice; and (iii) further adapting those selected strains for enhanced growth by additional passage from one to several times in the cell line(s) of choice. The selected viruses (for example, chosen from the four serotypes of Dengue viruses) can be further adapted to grow to high titers by additional cell culture passages or by genetic manipulation to make high-titered master and production seed lots.

Methods for producing Dengue virus(es) are known in the art, and are described in detail sufficient to guide one of ordinary skill in the art in, e.g., published PCT Application No. WO 2010/094663, US publication No. 2011318407. Methods for producing virus in serum-free conditions can also be found, for example, in US Publication No. 20060183224. The disclosures of these published patent applications are incorporated herein by reference to provide additional details regarding the propagation and purification of Dengue viruses for inclusion in the bulk preparations and immunogenic compositions disclosed herein. Similarly methods for inactivating Dengue viruses to produce a purified inactivated Dengue virus are well established in the art, and include exposure to chemical, physical and/or irradiating agents. Suitable methods include, for example, exposure to formaldehyde, betapropiolactone (BPL), hydrogen peroxide, ultraviolet irradiation and gamma irradiation, or combinations thereof. Details of such methods can be found, e.g., in published PCT Application No. WO 2010/094663 (US Publication No. 2011318407), and in US Publication No. 20070031451, which are incorporated herein by reference for the purpose of illustrating exemplary methods of inactivating Dengue viruses.

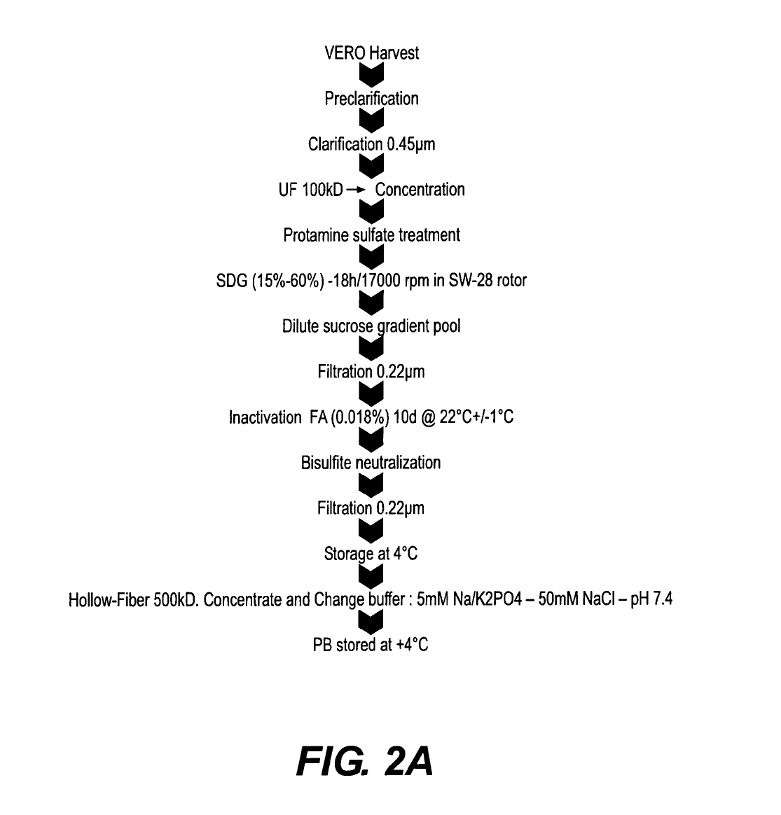

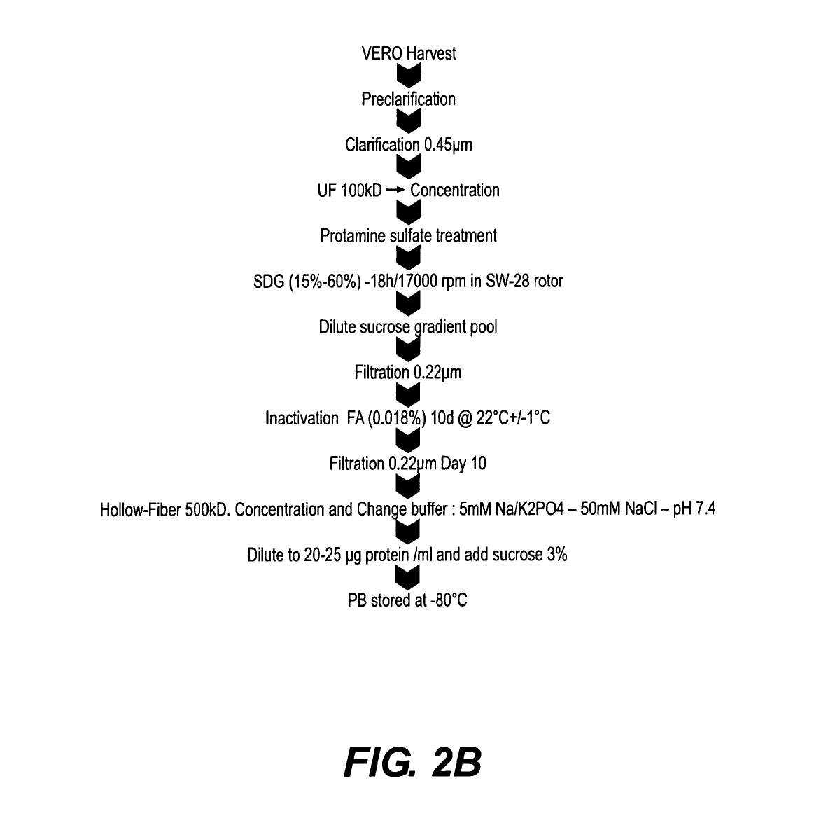

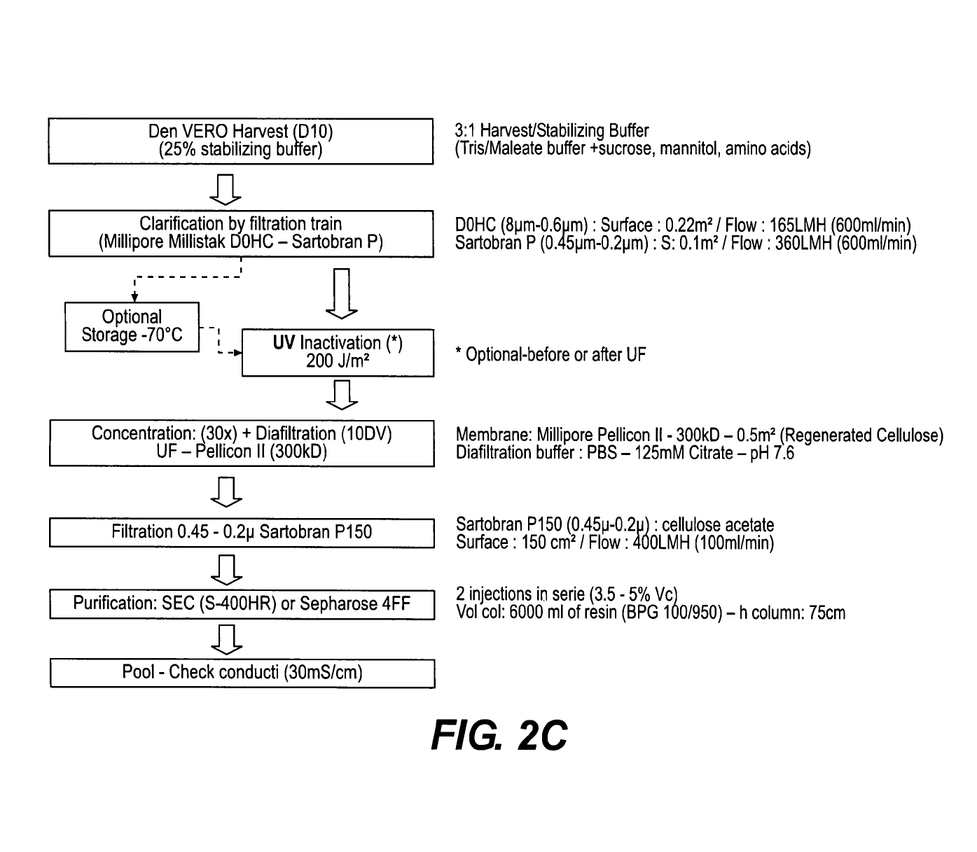

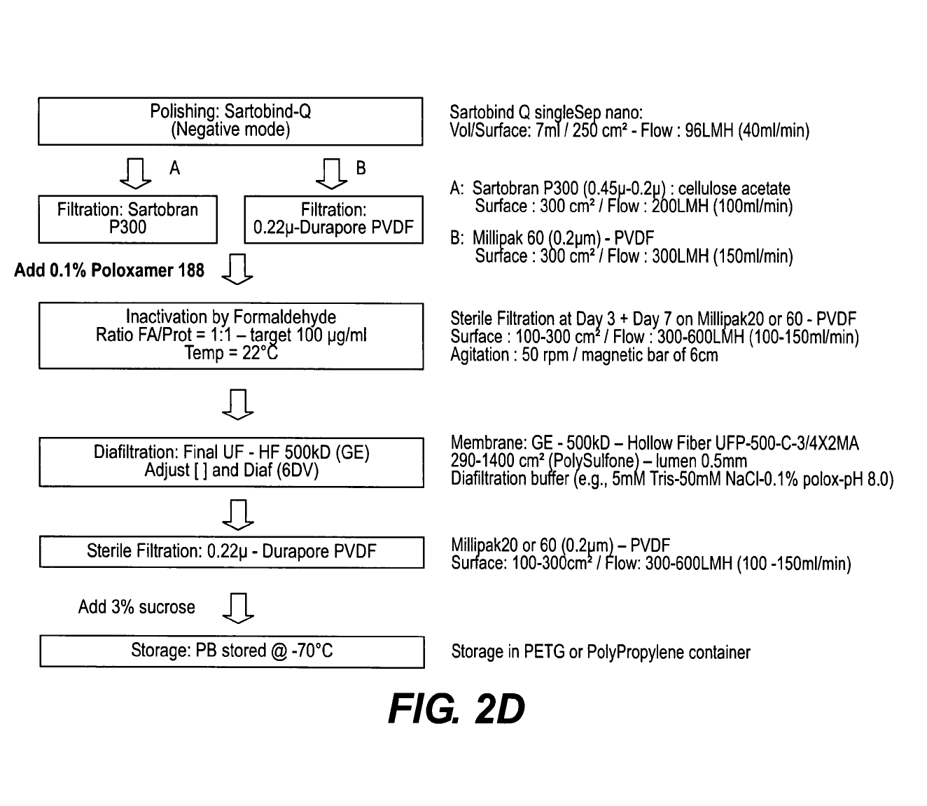

Exemplary procedures for purification of Dengue virus are depicted in the flow charts of FIGS. 2A-D. To produce quantities of Dengue virus suitable for commercial use, a susceptible cell line is grown in culture in vitro in a suitable medium. Typically the cells are mammalian cells, such as kidney or lung epithelial cells. Several suitable cell lines exist, e.g., African Green Monkey Kidney cells, such as Vero cells, MRC-5 cells, MDCK cells, and FRhL-2 cells. Alternatively, insect cells, particularly mosquito cells, such as the Aedes albopictus line C6/36 can be used. The cells can be cultured in either serum-containing or animal free (AF medium). Optionally, the medium is supplemented initially or periodically with additives, such as glucose, amino acids, synthetic growth factors or other proteins. The cells are expanded, typically through a sequence of increasing vessel size (e.g., 175 cm² flask; CF₂ (1200 cm²); CF₁₀ (6000 cm²); CF₄₀ (50 L bioreactor); 200 L bioreactor). In the larger vessel size, it is common to employ microcarriers in suspension for cell attachment. Optionally, the medium is supplied by perfusion, or the cultures can be fed periodically. In certain embodiments, the cells are Vero cells, which can be cultured in commercial scale bioreactors.

The cells are grown to desired density at scale and infected with the virus (e.g., strains selected to provide antigenic determinants of DEN-1, DEN-2, DEN-3 and/or DEN-4). The cells are infected at suitable MOI (e.g., 0.01-0.1 MOI, for example 0.05 MOI) with the selected. When employing serum-containing medium for preculture and/or infection, the medium can be exchanged for AF medium to reduce extraneous protein content during the harvest and purification phase. For example, after an initial infection phase of 1 to 4 days, e.g., approximately 2 days, the medium can be exchanged to AF medium. Optionally, the AF medium is initially or periodically supplemented with glucose, amino acids or the like. After a suitable period for viral growth, for example, between a minimum of 6 and 8 days, virus is harvested from the cells. Optionally, virus can be harvested incrementally at intervals (for example intervals of 2 days) starting at approximately 6 days post-infection. Harvest may favorably continue for a period of several days, for example up to day 10, such as until day 12, or day 14, or longer.

The medium containing the virus is clarified, typically through a series of decreasing pore sizes (e.g., 8 g, 0.6 g, 0.45 g, 0.2 g). Suitable commercially available filters and filtration devices are well known in the art and can be selected by those of skill. Exemplary filtration devices include, e.g., Millipore™ Millistak™ DOHC and Sartobran™ P filtration devices. Optionally, the clarified virus harvest can be stored frozen at −70° C. if desired.

The virus suspension is then concentrated (e.g., 20-50× or more, such as 30× or 40×) and the medium is exchanged for a suitable buffer (for example, phosphate buffered saline (PBS), 125 mM Citrate, pH 7.6), e.g., by ultrafiltration and diafiltration. The buffers selected at this stage and throughout purification are chosen to maintain pH, reduce aggregation and preserve antigenicity of the virus during processing. The buffers indicated herein are examples only, and alternative buffer solutions for the purposes indicated can be selected by those of skill in the art. Initial concentration and buffer exchange is followed by further filtration and size exclusion chromatography (SEC), using, e.g., Sephacryl S-400HR or Sepharose 4 FF resins. Optionally, prior to further processing, the clarified virus suspension is inactivated by exposure to UV irradiation (between 100-500, e.g., 200 J/m²), either before or after the concentration step.

Optionally, the size exclusion chromatography step can be followed by one or more steps, to remove residual nucleic acids, such as cellular DNA. For this purpose, one suitable method is membrane chromatography, e.g., Sartobind-Q membrane chromatography (in negative mode) and filtration. It is generally preferred that residual DNA be reduced to less than or equal to 100 pg DNA per μg protein (or to less than 100 pg/dose).

Favorably, at this stage, prior to inactivation, a surfactant, such as a Poloxamer surfactant as disclosed herein, and selected for inclusion in the bulk preparation and/or immunogenic composition can be added to the buffer. Alternatively, the surfactant can be added to buffer following inactivation. The virus is then inactivated, by any of one or more methods known in the art, including by chemical inactivation and/or by irradiation. Chemical inactivation, e.g., by formaldehyde, betapropiolactone (BPL) or by Hydrogen Peroxide have been described in the art for the inactivation of Dengue virus, and can be employed to provide purified inactivated Dengue virus in the context of the formulations disclosed herein. For example, the virus can be inactivated by exposure to formaldehyde (at approximately 100 μg/ml) for a period typically between 7 and 10 days at room temperature. Optionally, the suspension is filtered (e.g., 0.22μ) at an intermediate time point during the inactivation process, such as at day 2, 3, 4, or 5, to remove aggregates and improve formaldehyde exposure. The chemical means of inactivation can be used singly or in combination. Alternatively, or in combination with one or more chemical means, the virus can be inactivated by irradiation (e.g., UV or gamma irradiation). The formaldehyde or other chemical inactivating compound is then removed or neutralized (e.g., in the case of formaldehyde, with sodium bisulfite). Ultrafiltration/Diafiltration can be employed to remove the chemical inactivation agent and place the purified virus in a suitable buffer for subsequent formulation. The purified inactivated Dengue virus is then finally sterile filtered to produce a bulk preparation of inactivated Dengue virus. Optionally, sucrose is added to the final formulation of the bulk preparation. If desired, the final bulk preparation can be stored frozen at, e.g., −70° C.

The selected purified inactivated viruses are formulated as described herein to produce bulk preparations and immunogenic compositions that are stable and immunogenic, and which can be produced at commercial scale without the substantial loss during lyophilization and reconstitution observed with previously available methods and formulations. The methods described above can result in a purified inactivated Dengue virus preparation that is at least 70% and typically at least 80% Dengue viral material. The preparations contain less than 25% and typically less than 20% host cell proteins. Furthermore, according to the methods described above, the recovery of purified inactivated Dengue virus is substantially enhanced, such that greater than 90% (or greater than 95%) of the viral material is recovered in the final preparation. That is, a loss of less than 10%, or even less than 5% of the viral material is observed following the final 0.2 g filtration of the inactivated purified bulk. Thus, the present disclosure provides, inter alia, a method for reducing at least one of nonspecific adsorption and/or aggregation of a purified inactivated Dengue virus and a method for enhancing recovery of an antigenically preserved inactivated Dengue virus by formulating the inactivated Dengue virus according to disclosed methods.

In certain embodiments, the one or more purified inactivated Dengue viruses are adsorbed onto an aluminum salt prior to admixing with the solution to produce a pre-adsorbed bulk preparation of inactivated Dengue virus. Dengue virus is combined in solution with an aluminum salt and allowed to contact the aluminum particles for such time as to permit adsorption of the inactivated virus to the aluminum particles. Suitable aluminum salts include aluminum hydroxide, aluminum phosphate, aluminum hydroxyphosphate, and potassium aluminum sulfate. Typically, each selected virus is independently adsorbed onto aluminum to permit empiric optimization of the virus:aluminum ratio. In one favorable example, each selected Dengue virus is singly adsorbed onto aluminum hydroxide to produce an alum adsorbed monobulk prior to subsequent formulation with the other components of the immunogenic composition. Alternatively, each of the selected Dengue viruses can be adsorbed onto aluminum phosphate or another pharmaceutically acceptable aluminum salt. If desired, the purified inactivated Dengue viruses can be combined in the desired ratio and then adsorbed as a mixture onto the selected aluminum salt. Alternatively, instead of pre-adsorption onto an aluminum salt, the purified inactivated Dengue virus can be resuspended as described below in a solution that contains the selected aluminum salt.

In the context of the formulations disclosed herein, the solution to which the purified inactivated Dengue virus (optionally pre-adsorbed onto an aluminum salt) is admixed contains a buffering agent. That is, the solution is a buffered solution capable of resisting changes in pH that might otherwise be caused by addition of other components to the formulation, or final preparation of immunogenic composition for administration, as discussed below.

Dengue virus is sensitive to acidic pH, and at acidic pH, important immunological epitopes can be lost, diminishing the capacity of the purified inactivated virus antigen to elicit an immune response. The buffering agent is therefore selected to maintain the pH at or near neutral, or at slightly basic pH. To improve the final pH in some formulations, the buffering agent is selected to promote a pH in the initial formulation (e.g., before the addition of certain components such as adjuvants that may have an acidic pH) that is higher than that desired in the final composition administered to the subject. Accordingly, a buffering agent (or combination of agents) is selected to maintain the pH at or above pH 6.4. More preferably, the buffering agent is selected to maintain the pH at or above pH 6.8, most preferably, the buffering agent is selected to maintain the pH at or above neutral, e.g., at or near physiological pH of 7.4, and in some instance at or above pH 7.5, such as at or above pH 8.0, or even pH 8.5.

Suitable buffering agents include carbonate, phosphate, citrate, lactate, gluconate and tartrate buffering agents, as well as more complex organic buffering agents. In certain examples, the buffering agent includes a phosphate buffering agent that contains sodium phosphate and/or potassium phosphate. Typically, such a buffering agent, or system, includes both sodium phosphate and potassium phosphate in a ration selected to achieve the desired pH. In another example, the buffering agent contains Tris(hydroxymethyl)aminomethane, or “Tris”, formulated to achieve the desired pH. Methods of formulating buffers to the desired pH are well known to those of skill in the art, and a suitable composition can be determined without undue experimentation based on the pH desired.

In the formulations of bulk preparations and immunogenic compositions disclosed herein, the solution containing the purified inactivated Dengue virus(es) also includes a surfactant. Numerous surfactants are known in the art, and can be used in pharmaceutical formulations. The surfactant in the context of the formulations disclosed herein is selected to retain the immunological properties (e.g., conformation and immunological epitopes) of the purified inactivated Dengue virus, whilst increasing stability of the formulation and enhancing recovery, e.g., by reducing aspecific adsorption and/or aggregation of the virus.

Surfactants are amphiphilic molecules with a predominantly hydrophilic “head” and a hydrophobic “tail”. Surfactants can be classified according to the composition of their head and tail portions. Based on the characteristics of their head portion, surfactants can be classified as: nonionic (no-charge) or ionic (charged). Ionic surfactants can be divided into anionic (negatively charged), cationic (positively charged), and amphoteric, e.g., zwitterionic (two oppositely charged groups). Surfactants can also be categorized by the composition of their tail portion. Suitable surfactants include those with hydrocarbon (e.g., arene, alkane, alkene, cycloalkane and alkyne) tails; alkyl ether tails, ethoxylated (polyethylene oxide) tails; propoxylated (polypropylene oxides) tails.

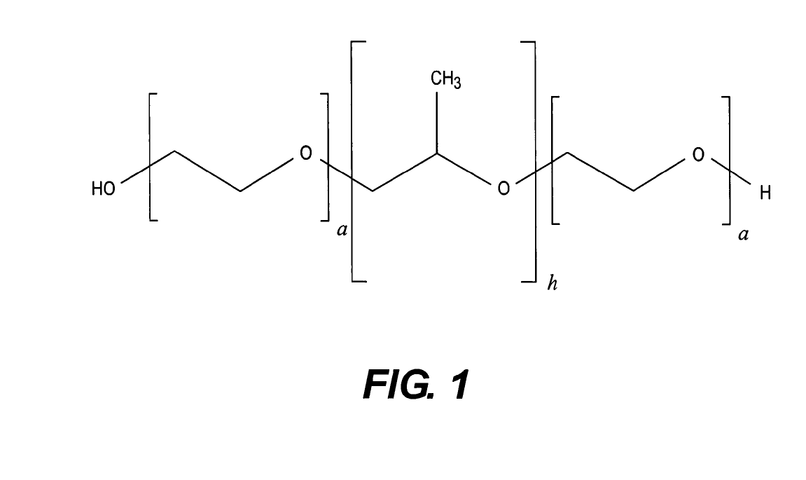

In certain embodiments, the selected surfactant is a zwitterionic surfactant. In an embodiment, the surfactant is an injectable surfactant. In the context of the instant formulations, one suitable class of surfactants includes the poloxamer surfactants. Poloxamers are nonionic triblock copolymers composed of a central hydrophobic chain of polyoxypropylene (polypropylene oxide) flanked by two hydrophilic chains of polyoxyethylene (polyethylene oxide), as illustrated schematically in FIG. 1. Poloxamers are also known by the trade name Pluronics™, and certain of these are sold under the trade name Lutrol™ or Kolliphor™. Poloxamer surfactants are particularly suitable for formulations in which the purified inactivated Dengue virus(es) are not adsorbed onto an aluminum salt.

In certain embodiments, the poloxamer surfactant is selected from a polyethylene-polypropylene glycol copolymer that is in solid form at room temperature, e.g., with an average molecular weight of at least about 4500 kD, and an average molecular weight of no more than about 15,000 kD. For example, the poloxamer surfactant can be selected from the group of Pluronic™ F108, Pluronic™ F127, Pluronic™ F188, Pluronic™ F38, Pluronic™ F68, Pluronic™ F77, Pluronic™ F87, Pluronic™ F88, and Pluronic™ F98. Various Pluronic™ surfactants are also sold under the trade name Lutrol™ (now Kolliphor™). In one specific embodiment, the immunogenic composition is formulated with a polyethylene-polypropylene glycol copolymer, designated Pluronic™ F 68 or Lutrol™ F 68 (Kolliphor™ P188), which has an average molecular weight of 8600 kD, with a polyoxypropylene molecular weight of 1800 g/mole and an 80% polyoxyethylene content. Alternatively, poloxamer surfactants that are in paste or liquid form at room temperature can be employed, e.g., having a molecular weight of at least about 1000 kD, such as Pluronic™ L 10, Pluronic™ L 101, Pluronic™ L 121, Pluronic™ L 31, Pluronic™ L 35, Pluronic™ L 43, Pluronic™ L 44, Pluronic™ L 61, Pluronic™ L 62, Pluronic™ L 64, Pluronic™ L 81, Pluronic™ L 92, Pluronic™ P 103, Pluronic™ P 104, Pluronic™ P 105, Pluronic™ P 123, Pluronic™ P 65, Pluronic™ P 84 or Pluronic™ P 85.

Other examples of suitable surfactants, in addition to poloxamer surfactants as noted above, in the context of the formulations disclosed herein include surfactants selected from the group consisting of a poloxamer, macrogol 15 hydroxy stearate, a polysorbate, a octoxinol, a polidocanol, a polyoxyl stearate, a polyoxyl castor oil, an N-octyl-glucoside, and combinations thereof.

The surfactant can be added to the formulation in an amount of at least 0.0001% and up to 1.0%. For example, the surfactant can be added in an amount of at least 0.0005% and up to 0.5%, such as between 0.001 and 0.2%, e.g., at a concentration of 0.0005%, or 0.001%, or 0.005%, or 0.01%, or 0.025%, or 0.05%, or 0.1%, or 0.2%, or 0.3%, or 0.4%, or 0.5%, or up to 1.0% (or any intervening amount). These concentrations are given as weight/volume in the initial formulation. It will be understood that in embodiments discussed below, in which the composition is lyophylized and/or lyophilized and resuspended, the precise amounts can be recalculated on a weight/weight basis (for solid compositions) and/or adjusted depending on the concentration or dilution factor of the final formulation to be administered to a subject.

Typically the final amount is calculated to be within the permissible daily exposure (PDE). For example, for Pluronic™ F68, the accepted PDE is 150 μg per dose injected. Accordingly, the concentration in the final formulation can vary depending on the volume to be administered to achieve the acceptable PDE.

In some embodiments, the formulations disclosed herein include an additional pharmaceutically acceptable component to modify tonicity, viscosity, stability, homogeneity or the like of the solution.

For example, the solution (and thus, the formulation) can include one or more salts. Most commonly, the salt is sodium chloride. However, other mineral salts and ions can also be used, e.g., salts of potassium, calcium, magnesium, manganese, zinc, as can other pharmaceutically acceptable salts and ions. Pharmaceutically acceptable salts and their selection are thoroughly discussed, e.g., in Pharmaceutical Salts: Properties, Selection, and Use, 2nd Revised Edition, P. Heinrich Stahl (Editor), Camille G. Wermuth (Editor), Wiley, 2011.

In some embodiments, the solution contains at least one additional excipient or carrier. For example, the solution (and thus, the formulation) can include at least one sugar or polyol (or combinations thereof), including carbohydrate and non-carbohydrate polyols, e.g., glass forming sugars and polyols. The excipient is typically selected to enable the inactivated Dengue virus to be stored without substantial loss of immunologically important epitopes. Examples of suitable excipients include sugars, sugar alcohols and carbohydrate derivatives.

Carbohydrates include, but are not limited to, monosaccharides, disaccharides, trisaccharides, oligosaccharides and their corresponding sugar alcohols, polyhydroxyl compounds such as carbohydrate derivatives and chemically modified carbohydrates, hydroxyethyl starch and sugar copolymers. Both natural and synthetic carbohydrates are suitable for use. Synthetic carbohydrates include, but are not limited to, those which have the glycosidic bond replaced by a thiol or carbon bond. Both D and L forms of the carbohydrates may be used. The carbohydrate may be non-reducing or reducing. Where a reducing carbohydrate is used, the addition of inhibitors of the Maillard reaction is preferred.

Reducing carbohydrates suitable for use in the invention are those known in the art and include, but are not limited to, glucose, maltose, lactose, fructose, galactoase, mannose, maltulose and lactulose. Non-reducing carbohydrates include, but are not limited to, non-reducing glycosides of polyhydroxyl compounds selected from sugar alcohols and other straight chain polyalcohols. Other useful carbohydrates include raffinose, stachyose, melezitose, dextran, sucrose, cellibiose, mannobiose and sugar alcohols. The sugar alcohol glycosides are preferably monoglycosides, in particular the compounds obtained by reduction of disaccharides such as lactose, maltose, lactulose and maltulose.

Typically, the excipient is selected from the group of carbohydrates (or derivative thereof) including glucose, maltulose, iso-maltulose, lactulose, lactobionic acid, sucrose, maltose, lactose, glucose, iso-maltose, mannitol, maltitol, lactitol, sorbitol, palatinit, trehalose, raffinose, stachyose, melezitose mannose or dextran, or a combination thereof. In certain examples, the glass forming sugar or polyol is selected from the group consisting of: sucrose, trehalose, mannose, mannitol, raffinose, lactitol, sorbitol and lactobionic acid, glucose, maltulose, iso-maltulose, lactulose, maltose, lactose, iso-maltose, maltitol, palatinit, stachyose, melezitose, dextran or a combination thereof. In one specific embodiment, the excipient is sucrose.

The concentration of the sugar or polyol included in the solution can be between 1% and 50% weight/volume, such as 1-10%, (e.g., 1-5%, 3-7%, 5-10%, or any intervening interval), or 10-15%, 15-20%, 20-25% or 25-50%, most preferably less than or equal to 5% or less than or equal to 10% (w/v).

Alternatively, or in addition, the excipient can include an amino acid, such as glycine, alanine, arginine, lysine and glutamine although any amino acid, or a combination of amino acids, peptide, hydrolysed protein or protein such as serum albumin can be included.

Exemplary Formulation Compositions are provided in Table 1.

| | pH |
|---|---|
| Buffer | (+/1 0.1) |
| 5 mM Na/K2PO4, 50 mM NaCl | 7.6 |
| 5 mM Na/K2PO4, 50 mM NaCl, 0.1% Poloxamer 188, | 7.6 |
| 3% Sucrose | |
| 10 mM Na/K2PO4, 0.1% Poloxamer 188, 3% Sucrose | 7.6 |
| 5 mM K/K2PO4, 10 mM Citrate, 0.1% Poloxamer 188, | 7.6 |
| 3% Sucrose | |
| PBS, 125 mM Citrate | 7.6 |
| 5 mM Tris, 5 mM Maleate, 0.1% Poloxamer 188, | 7.5 |
| 3% Sucrose | |
| 5 mM Tris, 50 mM NaCl, 0.1% Poloxamer 188, | 8.0 |
| 3% Sucrose | |
| 5 mM Tris, 5 mM Maleate, 50 mM NaCl0.1% | 7.5 |
| Poloxamer 188, 3% Sucrose | |
| 10 mM Na/K2PO4, 0.1% Poloxamer 188, 1% Sorbitol | 7.6 |
| 10 mM K/K2PO4, 0.4% Histidine, 0.1% | 7.6 |
| Poloxamer 188, 1% Sorbitol | |

In one favorable embodiment, the buffer comprises 5 mM Tris, 50 mM NaCl, optionally with a surfactant, e.g., Poloxamer 188 and a sugar, e.g., sucrose. Nonetheless, it will be appreciated that the buffer examples provided herein are not intended to be limiting, either by the specific components, or by the specific combinations provided as examples.

Typically, the solution in which the purified inactivated Dengue virus(es) are formulated is prepared by adding the various components to endotoxin-free water (e.g., sterile water). For example, the solution to which the purified inactivated Dengue virus(es) are added can be prepared by adding to endotoxin-free water: a glass forming sugar or polyol; a buffering agent; a salt; and a surfactant. In an embodiment, the components are added sequentially in the order: a glass forming sugar or polyol; a buffering agent; a salt; and a surfactant. The components can be sterile, and/or the solution can be sterilized, e.g., by filtration or other convenient methods. In the event that the purified inactivated virus(es) is in a solution that contains one or more of the components to be included in the final formulation, the amount can be adjusted to the selected concentration of the final bulk preparation or immunogenic composition.

Additional pharmaceutically acceptable carriers and excipients may also be included in the formulation, such carriers and excipients are well known in the art, and are described, e.g., in Remington's Pharmaceutical Sciences, by E. W. Martin, Mack Publishing Co., Easton Pa., 5^{th} Ed.

In certain embodiments, following addition of the purified inactivated Dengue virus to the solution containing the buffer and the surfactant (and optionally, additional components) as described above, the formulated immunogenic composition is stored as a liquid, e.g., at room temperature, at 0-4° C., or below 0° C., such as at or about −20° C., or at or about −70 to −80° C.

Alternatively, the formulated composition is dried, e.g., by lyophilization to produce a dried or lyophilized composition. Drying (by evaporation of the solvent from the formulation) can be accomplished by lyophilization. Lyophilization is performed on a solvent/solute mixture under a vacuum resulting in the sublimation of the solvent, and leaving behind the dried solute(s), including the purified inactivated Dengue virus(es) and other components of the formulation. Any pressure less than 100 μbar is likely to be suitable. Typically a vacuum of at least about 500 mBar is sufficient to promote efficient evaporation of a solvent, and a vacuum of at least about 6 mBar is sufficient to promote efficient sublimation of a solvent. Although the pressure can be further reduced, doing so has little effect on drying rate, and under very low pressure conditions, efficiency of sublimation is decreased. Although solvent removal can be performed simply by placing a liquid sample into a vacuum chamber, foaming or frothing can result in product loss, as well as decreases in product homogeneity or immunogenicity. To prevent foaming or frothing, the formulated immunogenic composition can first be frozen and solvent can then be removed by sublimation under vacuum, i.e., by lyophilization or freeze drying. An exemplary procedure is outlined in Example 3.

Thus, in certain embodiments, this disclosure provides lyophilized preparations of inactivated Dengue virus that contains at least one purified inactivated Dengue virus and a surfactant, such as the surfactants disclosed above, e.g., poloxamer surfactants, etc. In some cases the lyophilized preparation contains an aluminum salt, such as aluminum hydroxide or aluminum phosphate. For example, the one or more than one purified inactivated Dengue virus can be adsorbed onto the aluminum salt. The lyophilized preparations can also include at least one component that acts as a buffering agent and/or at least one of a glass forming sugar and a glass forming polyol. One of ordinary skill in the art will recognize further embodiments and alternatives based on the disclosures above.

In embodiments where the formulated composition is dried, the dried composition is typically resuspended in a pharmaceutically acceptable solvent prior to administration, e.g., as an injectable liquid. The solution to which the purified inactivated Dengue virus is admixed is selected to be suitable for pharmaceutical administration to a human subject. Typically, the solution is chosen to be acceptable for parenteral administration, e.g., by intramuscular, subcutaneous, transcutaneous or intradermal administration. For example, the dried composition can be resuspended in water for injection, e.g., sterile, endotoxin-free water. Alternatively, the solvent may be a mixture of aqueous and organic solvents. In some embodiments, the resuspended immunogenic compositions are isotonic. Alternatively, if the resuspended immunogenic composition is not isotonic, the tonicity can be adjusted, e.g., by the addition of a salt or other excipient, to isotonic or near isotonic prior to administration. It will be appreciated by those of ordinary skill in the art that the volumes, before and optionally after lyophilization, where relevant, can be selected and adjusted based on convenience. Depending on the relative volumes, e.g., of the formulate immunogenic composition prior to lyophilization and following resupension, the final composition prepared for administration can be in a lesser or greater volume, and therefore can be more or less concentrated than the formulation exemplified herein. Such adjustments in concentration can be readily calculated without undue experimentation to suit preference.

Typically, the amount of virus in each dose of immunogenic composition is selected as an amount that induces an immunoprotective response (following one or more doses) without significant, adverse side effects in the typical subject. Immunoprotective in this context does not necessarily mean completely protective against infection; it means protection against symptoms or disease, especially severe disease associated with the virus. The amount of antigen can vary depending upon which specific immunogen is employed. Antigen content can be measured in terms of μg total protein content of a purified or partially purified virus antigen, or by immunological methods, e.g., ELISA, or by a quantitative immunoprecipitation method such as radial immunodiffusion. Generally, it is expected that each human dose will comprise 0.01-100 μg of inactivated virus, such as at least about 0.1 μg (e.g., 0.1, 0.2, 0.25, 0.3, 0.33, 0.4, or 0.5 μg) to no more than about 50 μg, for example, from about 0.25 μg to about 30 μg, such as about 0.25 μg, 0.33 μg, 0.5 μg, 1 μg, about 2 μg, about 2.5 μg, about 3 μg, about 4 μg, about 5 μg, or about 10 μg (or any amount between 0.1 and 10.0 μg) of each serotype of virus. Typically, a single human dose of the immunogenic composition contains no more than about 100 μg, for example, no more than about 90 μg, or no more than about 80 μg, or no more than about 75 μg, or no more than about 70 μg, or no more than about 60 μg, or no more than about 50 μg, or no more than 40 μg, or no more than 30 μg, or no more than 20 μg, or no more than 10 μg (or any amount between 10 and 100 μg) of each serotype of virus. For example, a single human dose of the immunogenic composition can include between 0.10 and 10 μg, or between 0.25 and 5 μg per human dose, or any other range defined by the individual parameters recited above.

The amount utilized in an immunogenic composition is selected based on the subject population (e.g., infant). An optimal amount for a particular composition can be ascertained by standard studies involving observation of antibody titres and other responses in subjects. Following an initial vaccination, subjects can receive one or more additional doses after a suitable interval (e.g., in about 4 weeks). Immunoprotection can typically result after at least two doses of an immunogenic composition as described herein, and in some instances results after two or three or more doses, delivered after suitable intervals.

In some embodiments, the immunogenic composition includes at least one immunostimulatory component or adjuvant. In some instances, the adjuvant comprises a mineral salt, such as an aluminum (alum) salt, for example potassium aluminum sulfate, aluminium phosphate or aluminium hydroxide. Where alum is present, the amount is typically between about 100 μg and 1 mg, such as from about 100 μg, or about 200 μg to about 750 μg, such as about 500 μg per dose. As discussed above, in formulations in which an aluminum salt is employed, the purified inactivated Dengue virus(es) can be pre-adsorbed onto the aluminum salt prior to formulation in the compositions disclosed herein. Alternatively, the aluminum salt can be included in the liquid in which the lyophilized immunogenic composition is resuspended, or added to the liquid composition. In addition to aluminum salts, calcium salts can also be employed, e.g., as particulate adjuvants.

Alternatively or in addition (e.g., to an aluminum salt), the liquid in which the dried formulation is resuspended can include an immunostimulatory component. The immunostimulatory component can also be added to a liquid formulation prior to administration (e.g., prepared in two vials and/or syringes or other containers, and mixed prior to administration). For example, when the immunogenic composition is formulated for intramuscular administration, adjuvants including one or more of 3D-MPL, squalene (e.g., QS21), liposomes, and/or oil and water emulsions are favorably selected.

One suitable adjuvant for use in combination with purified inactivated Dengue virus antigens is a non-toxic bacterial lipopolysaccharide derivative. An example of a suitable non-toxic derivative of lipid A, is monophosphoryl lipid A or more particularly 3-Deacylated monophoshoryl lipid A (3D-MPL). 3D-MPL is sold under the name MPL by GlaxoSmithKline Biologicals N. A., and is referred throughout the document as MPL or 3D-MPL. See, for example, U.S. Pat. Nos. 4,436,727; 4,877,611; 4,866,034 and 4,912,094. 3D-MPL primarily promotes CD4+ T cell responses with an IFN-γ (Th1) phenotype. 3D-MPL can be produced according to the methods disclosed in GB2220211 A. Chemically it is a mixture of 3-deacylated monophosphoryl lipid A with 3, 4, 5 or 6 acylated chains. In the compositions of the present invention small particle 3D-MPL can be used. Small particle 3D-MPL has a particle size such that it can be sterile-filtered through a 0.22 μm filter. Such preparations are described in WO94/21292.

A lipopolysaccharide, such as 3D-MPL, can be used at amounts between 1 and 50 μg, per human dose of the immunogenic composition. Such 3D-MPL can be used at a level of about 25 μg, for example between 20-30 μg, suitably between 21-29 μg or between 22 and 28 μg or between 23 and 27 μg or between 24 and 26 μg, or 25 μg. In another embodiment, the human dose of the immunogenic composition comprises 3D-MPL at a level of about 10 μg, for example between 5 and 15 μg, suitably between 6 and 14 μg, for example between 7 and 13 μg or between 8 and 12 μg or between 9 and 11 μg, or 10 μg. In a further embodiment, the human dose of the immunogenic composition comprises 3D-MPL at a level of about 5 μg, for example between 1 and 9 μg, or between 2 and 8 μg or suitably between 3 and 7 μg or 4 and 6 μg, or 5 μg.

In other embodiments, the lipopolysaccharide can be a β(1-6) glucosamine disaccharide, as described in U.S. Pat. No. 6,005,099 and EP Patent No. 0 729 473 B1. One of skill in the art would be readily able to produce various lipopolysaccharides, such as 3D-MPL, based on the teachings of these references. Nonetheless, each of these references is incorporated herein by reference. In addition to the aforementioned immunostimulants (that are similar in structure to that of LPS or MPL or 3D-MPL), acylated monosaccharide and disaccharide derivatives that are a sub-portion to the above structure of MPL are also suitable adjuvants. In other embodiments, the adjuvant is a synthetic derivative of lipid A, some of which are described as TLR-4 agonists, and include, but are not limited to: OM174 (2-deoxy-6-o-[2-deoxy-2-[(R)-3-dodecanoyloxytetra-decanoylamino]-4-o-phosphono-β-D-glucopyranosyl]-2-[(R)-3-hydroxytetradecanoylamino]-α-D-glucopyranosyldihydrogenphosphate), (WO 95/14026); OM 294 DP (3S,9R)-3-[(R)-dodecanoyloxytetradecanoylamino]-4-oxo-5-aza-9(R)-[(R)-3-hydroxytetradecanoylamino]decan-1,10-diol,1,10-bis(dihydrogenophosphate) (WO 99/64301 and WO 00/0462); and OM 197 MP-Ac DP (3S—,9R)-3-[(R)-dodecanoyloxytetradecanoylamino]-4-oxo-5-aza-9-[(R)-3-hydroxytetradecanoylamino]decan-1,10-diol,1-dihydrogenophosphate 10-(6-aminohexanoate) (WO 01/46127).

Other immunostimulatory components that can be used in immunogenic compositions with purified inactivated Dengue virus(es), e.g., on their own or in combination with 3D-MPL, or another adjuvant described herein, are saponins, such as QS21.

Saponins are taught in: Lacaille-Dubois, M and Wagner H. (1996. A review of the biological and pharmacological activities of saponins. Phytomedicine vol 2 pp 363-386). Saponins are steroid or triterpene glycosides widely distributed in the plant and marine animal kingdoms. Saponins are noted for forming colloidal solutions in water which foam on shaking, and for precipitating cholesterol. When saponins are near cell membranes they create pore-like structures in the membrane which cause the membrane to burst. Haemolysis of erythrocytes is an example of this phenomenon, which is a property of certain, but not all, saponins.

Saponins are known as adjuvants in vaccines for systemic administration. The adjuvant and haemolytic activity of individual saponins has been extensively studied in the art (Lacaille-Dubois and Wagner, supra). For example, Quil A (derived from the bark of the South American tree Quillaja Saponaria Molina), and fractions thereof, are described in U.S. Pat. No. 5,057,540 and “Saponins as vaccine adjuvants”, Kensil, C. R., Crit. Rev Ther Drug Carrier Syst, 1996, 12 (1-2):1-55; and EP 0 362 279 B1. Particulate structures, termed Immune Stimulating Complexes (ISCOMS), comprising fractions of Quil A are haemolytic and have been used in the manufacture of vaccines (Morein, B., EP 0 109 942 B1; WO 96/11711; WO 96/33739). The haemolytic saponins QS21 and QS17 (HPLC purified fractions of Quil A) have been described as potent systemic adjuvants, and the method of their production is disclosed in U.S. Pat. No. 5,057,540 and EP 0 362 279 B1, which are incorporated herein by reference. Other saponins which have been used in systemic vaccination studies include those derived from other plant species such as Gypsophila and Saponaria (Bomford et al., Vaccine, 10(9):572-577, 1992). QS21 is an Hplc purified non-toxic fraction derived from the bark of Quillaja Saponaria Molina. A method for producing QS21 is disclosed in U.S. Pat. No. 5,057,540. Non-reactogenic adjuvant formulations containing QS21 are described in WO 96/33739. The aforementioned references are incorporated by reference herein. Said immunologically active saponin, such as QS21, can be used in amounts of between 1 and 50 μg, per human dose of the immunogenic composition. Advantageously QS21 is used at a level of about 25 μg, for example between 20-30 μg, suitably between 21-29 μg or between 22-28 μg or between 23-27 μg or between 24-26 μg, or 25 μg. In another embodiment, the human dose of the immunogenic composition comprises QS21 at a level of about 10 μg, for example between 5 and 15 μg, suitably between 6-14 μg, for example between 7-13 μg or between 8-12 μg or between 9-11 μg, or 10 μg. In a further embodiment, the human dose of the immunogenic composition comprises QS21 at a level of about 5 μg, for example between 1-9 μg, or between 2-8 μg or suitably between 3-7 μg or 4-6 μg, or 5 μg. Such formulations comprising QS21 and cholesterol have been shown to be successful Th1 stimulating adjuvants when formulated together with an antigen. Thus, for example, purified inactivated Dengue virus(es) can favorably be employed in immunogenic compositions with an adjuvant comprising a combination of QS21 and cholesterol.

Other TLR4 ligands which can be used are alkyl Glucosaminide phosphates (AGPs) such as those disclosed in WO 98/50399 or U.S. Pat. No. 6,303,347 (processes for preparation of AGPs are also disclosed), suitably RC527 or RC529 or pharmaceutically acceptable salts of AGPs as disclosed in U.S. Pat. No. 6,764,840. Some AGPs are TLR4 agonists, and some are TLR4 antagonists. Both are thought to be useful as adjuvants.

Other suitable TLR-4 ligands, capable of causing a signaling response through TLR-4 (Sabroe et al, JI 2003 p1630-5) are, for example, lipopolysaccharide from gram-negative bacteria and its derivatives, or fragments thereof, in particular a non-toxic derivative of LPS (such as 3D-MPL). Other suitable TLR agonists are: heat shock protein (HSP) 10, 60, 65, 70, 75 or 90; surfactant Protein A, hyaluronan oligosaccharides, heparan sulphate fragments, fibronectin fragments, fibrinogen peptides and b-defensin-2, and muramyl dipeptide (MDP). In one embodiment the TLR agonist is HSP 60, 70 or 90. Other suitable TLR-4 ligands are as described in WO 2003/011223 and in WO 2003/099195, such as compound I, compound II and compound III disclosed on pages 4-5 of WO2003/011223 or on pages 3-4 of WO2003/099195 and in particular those compounds disclosed in WO2003/011223 as ER803022, ER803058, ER803732, ER804053, ER804057, ER804058, ER804059, ER804442, ER804680, and ER804764. For example, one suitable TLR-4 ligand is ER804057.

Additional TLR agonists are also useful as adjuvants. The term “TLR agonist” refers to an agent that is capable of causing a signaling response through a TLR signaling pathway, either as a direct ligand or indirectly through generation of endogenous or exogenous ligand. Such natural or synthetic TLR agonists can be used as alternative or additional adjuvants. A brief review of the role of TLRs as adjuvant receptors is provided in Kaisho & Akira, Biochimica et Biophysica Acta 1589:1-13, 2002. These potential adjuvants include, but are not limited to agonists for TLR2, TLR3, TLR7, TLR8 and TLR9. Accordingly, in one embodiment, the adjuvant and immunogenic composition further comprises an adjuvant which is selected from the group consisting of: a TLR-1 agonist, a TLR-2 agonist, TLR-3 agonist, a TLR-4 agonist, TLR-5 agonist, a TLR-6 agonist, TLR-7 agonist, a TLR-8 agonist, TLR-9 agonist, or a combination thereof.

In one embodiment of the present invention, a TLR agonist is used that is capable of causing a signaling response through TLR-1. Suitably, the TLR agonist capable of causing a signaling response through TLR-1 is selected from: Tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorferi.

In an alternative embodiment, a TLR agonist is used that is capable of causing a signaling response through TLR-2. Suitably, the TLR agonist capable of causing a signaling response through TLR-2 is one or more of a lipoprotein, a peptidoglycan, a bacterial lipopeptide from M. tuberculosis, B burgdorferi or T. pallidum; peptidoglycans from species including Staphylococcus aureus; lipoteichoic acids, mannuronic acids, Neisseria porins, bacterial fimbriae, Yersina virulence factors, CMV virions, measles haemagglutinin, and zymosan from yeast.

In an alternative embodiment, a TLR agonist is used that is capable of causing a signaling response through TLR-3. Suitably, the TLR agonist capable of causing a signaling response through TLR-3 is double stranded RNA (dsRNA), or polyinosinic-polycytidylic acid (Poly IC), a molecular nucleic acid pattern associated with viral infection.

In an alternative embodiment, a TLR agonist is used that is capable of causing a signaling response through TLR-5. Suitably, the TLR agonist capable of causing a signaling response through TLR-5 is bacterial flagellin.

In an alternative embodiment, a TLR agonist is used that is capable of causing a signaling response through TLR-6. Suitably, the TLR agonist capable of causing a signaling response through TLR-6 is mycobacterial lipoprotein, di-acylated LP, and phenol-soluble modulin. Additional TLR6 agonists are described in WO 2003/043572.

In an alternative embodiment, a TLR agonist is used that is capable of causing a signaling response through TLR-7. Suitably, the TLR agonist capable of causing a signaling response through TLR-7 is a single stranded RNA (ssRNA), loxoribine, a guanosine analogue at positions N7 and C8, or an imidazoquinoline compound, or derivative thereof. In one embodiment, the TLR agonist is imiquimod. Further TLR7 agonists are described in WO 2002/085905.

In an alternative embodiment, a TLR agonist is used that is capable of causing a signaling response through TLR-8. Suitably, the TLR agonist capable of causing a signaling response through TLR-8 is a single stranded RNA (ssRNA), an imidazoquinoline molecule with anti-viral activity, for example resiquimod (R848); resiquimod is also capable of recognition by TLR-7. Other TLR-8 agonists which can be used include those described in WO 2004/071459.

In an alternative embodiment, a TLR agonist is used that is capable of causing a signaling response through TLR-9. In one embodiment, the TLR agonist capable of causing a signaling response through TLR-9 is HSP90. Alternatively, the TLR agonist capable of causing a signaling response through TLR-9 is bacterial or viral DNA, DNA containing unmethylated CpG nucleotides, in particular sequence contexts known as CpG motifs. CpG-containing oligonucleotides induce a predominantly Th1 response. Such oligonucleotides are well known and are described, for example, in WO 96/02555, WO 99/33488 and U.S. Pat. Nos. 6,008,200 and 5,856,462. Suitably, CpG nucleotides are CpG oligonucleotides. Suitable oligonucleotides for use in the immunogenic compositions of the present invention are CpG containing oligonucleotides, optionally containing two or more dinucleotide CpG motifs separated by at least three, suitably at least six or more nucleotides. A CpG motif is a Cytosine nucleotide followed by a Guanine nucleotide. The CpG oligonucleotides of the present invention are typically deoxynucleotides. In a specific embodiment the internucleotide in the oligonucleotide is phosphorodithioate, or suitably a phosphorothioate bond, although phosphodiester and other internucleotide bonds are within the scope of the invention. Also included within the scope of the invention are oligonucleotides with mixed internucleotide linkages. Methods for producing phosphorothioate oligonucleotides or phosphorodithioate are described in U.S. Pat. Nos. 5,666,153, 5,278,302 and WO 95/26204.

Another class of adjuvants for use in formulations with purified inactivated Dengue virus(es) includes OMP-based immunostimulatory compositions. OMP-based immunostimulatory compositions are particularly suitable as mucosal adjuvants, e.g., for intranasal administration. OMP-based immunostimulatory compositions are a genus of preparations of outer membrane proteins (OMPs, including some porins) from Gram-negative bacteria, such as, but not limited to, Neisseria species (see, e.g., Lowell et al., J. Exp. Med. 167:658, 1988; Lowell et al., Science 240:800, 1988; Lynch et al., Biophys. J. 45:104, 1984; Lowell, in “New Generation Vaccines” 2nd ed., Marcel Dekker, Inc., New York, Basil, Hong Kong, page 193, 1997; U.S. Pat. No. 5,726,292; U.S. Pat. No. 4,707,543), which are useful as a carrier or in compositions for immunogens, such as bacterial or viral antigens. Some OMP-based immunostimulatory compositions can be referred to as “Proteosomes,” which are hydrophobic and safe for human use. Proteosomes have the capability to auto-assemble into vesicle or vesicle-like OMP clusters of about 20 nm to about 800 nm, and to noncovalently incorporate, coordinate, associate (e.g., electrostatically or hydrophobically), or otherwise cooperate with protein antigens (Ags), particularly antigens that have a hydrophobic moiety. Any preparation method that results in the outer membrane protein component in vesicular or vesicle-like form, including multi-molecular membranous structures or molten globular-like OMP compositions of one or more OMPs, is included within the definition of Proteosome. Proteosomes can be prepared, for example, as described in the art (see, e.g., U.S. Pat. No. 5,726,292 or U.S. Pat. No. 5,985,284). Proteosomes can also contain an endogenous lipopolysaccharide or lipooligosaccharide (LPS or LOS, respectively) originating from the bacteria used to produce the OMP porins (e.g., Neisseria species), which generally will be less than 2% of the total OMP preparation.

Proteosomes are composed primarily of chemically extracted outer membrane proteins (OMPs) from Neisseria menigitidis (mostly porins A and B as well as class 4 OMP), maintained in solution by detergent (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206). Proteosomes can be formulated with a variety of antigens such as purified or recombinant proteins derived from viral sources, including the PreF polypeptides disclosed herein, e.g., by diafiltration or traditional dialysis processes. The gradual removal of detergent allows the formation of particulate hydrophobic complexes of approximately 100-200 nm in diameter (Lowell G H. Proteosomes for Improved Nasal, Oral, or Injectable Vaccines. In: Levine M M, Woodrow G C, Kaper J B, Cobon G S, eds, New Generation Vaccines. New York: Marcel Dekker, Inc. 1997; 193-206).

“Proteosome: LPS or Protollin” as used herein refers to preparations of proteosomes admixed, e.g., by the exogenous addition, with at least one kind of lipo-polysaccharide to provide an OMP-LPS composition (which can function as an immunostimulatory composition). Thus, the OMP-LPS composition can be comprised of two of the basic components of Protollin, which include (1) an outer membrane protein preparation of Proteosomes (e.g., Projuvant) prepared from Gram-negative bacteria, such as Neisseria meningitidis, and (2) a preparation of one or more liposaccharides. A lipo-oligosaccharide can be endogenous (e.g., naturally contained with the OMP Proteosome preparation), can be admixed or combined with an OMP preparation from an exogenously prepared lipo-oligosaccharide (e.g., prepared from a different culture or microorganism than the OMP preparation), or can be a combination thereof. Such exogenously added LPS can be from the same Gram-negative bacterium from which the OMP preparation was made or from a different Gram-negative bacterium. Protollin should also be understood to optionally include lipids, glycolipids, glycoproteins, small molecules, or the like, and combinations thereof. The Protollin can be prepared, for example, as described in U.S. Patent Application Publication No. 2003/0044425.

Combinations of different adjuvants, such as those mentioned hereinabove, can also be used in compositions with purified inactivated Dengue virus(es). For example, as already noted, QS21 can be formulated together with 3D-MPL. The ratio of QS21:3D-MPL will typically be in the order of 1:10 to 10:1; such as 1:5 to 5:1, and often substantially 1:1. Typically, the ratio is in the range of 2.5:1 to 1:1 3D-MPL to QS21. Optionally, such a combination can be in the form of a liposome.

Another combination adjuvant formulation includes 3D-MPL and an aluminium salt, such as aluminium hydroxide. When formulated in combination, this combination can enhance an antigen-specific Th1 immune response.

In some embodiments, the adjuvant includes an oil and water emulsion, e.g., an oil-in-water emulasion. One example of an oil-in-water emulsion comprises a metabolisable oil, such as squalene, and a surfactant, such as sorbitan trioleate (Span 85™) or polyoxyethylene sorbitan monooleate (Tween 80™), or a combination thereof, in an aqueous carrier. The aqueous carrier can be, for example, phosphate buffered saline. In certain embodiments, the oil-in-water emulsion does not contain any additional immunostimulants(s), (in particular it does not contain a non-toxic lipid A derivative, such as 3D-MPL, or a saponin, such as QS21). In certain embodiments, the oil-in-water emulsion includes a tocol such as a tocopherol, e.g., alpha-tocopherol. Additionally the oil-in-water emulsion can contain lecithin and/or tricaprylin.

In another embodiment of the invention there is provided a vaccine composition comprising an antigen or antigen composition and an adjuvant composition comprising an oil-in-water emulsion and optionally one or more additional immunostimulants, wherein said oil-in-water emulsion comprises 0.5-10 mg metabolisable oil (suitably squalene), 0.4-4 mg emulsifying agent, and optionally 0.5-11 mg tocol (suitably a tocopherol, such as alpha-tocopherol).

In one specific embodiment, the adjuvant formulation includes 3D-MPL prepared in the form of an emulsion, such as an oil-in-water emulsion. In some cases, the emulsion has a small particle size of less than 0.2 μm in diameter, as disclosed in WO 94/21292. For example, the particles of 3D-MPL can be small enough to be sterile filtered through a 0.22 micron membrane (as described in European Patent number 0 689 454). Alternatively, the 3D-MPL can be prepared in a liposomal formulation. Optionally, the adjuvant containing 3D-MPL (or a derivative thereof) also includes an additional immunostimulatory component.

The adjuvant is selected to be safe and effective in the population to which the immunogenic composition is administered. For adult and elderly populations, the formulations typically include more of an adjuvant component than is typically found in an infant formulation. When an immunogenic composition with a purified inactivated Dengue virus is formulated for administration to an infant, the dosage of adjuvant is determined to be effective and relatively non-reactogenic in an infant subject. Generally, the dosage of adjuvant in an infant formulation is lower (for example, the dose may be a fraction of the dose provided in a formulation to be administered to adults) than that used in formulations designed for administration to adult (e.g., adults aged 65 or older). For example, the amount of 3D-MPL is typically in the range of 1 μg-200 μg, such as 10-100 μg, or 10 μg-50 μg per dose. An infant dose is typically at the lower end of this range, e.g., from about 1 μg to about 50 μg, such as from about 2 μg, or about 5 μg, or about 10 μg, to about 25 μg, or to about 50 μg. Typically, where QS21 is used in the formulation, the ranges are comparable (and according to the ratios indicated above). In the case of an oil and water emulsion (e.g., an oil-in-water emulsion), the dose of adjuvant provided to a child or infant can be a fraction of the dose administered to an adult subject.

Thus, the formulated immunogenic composition (including any adjuvant) is suitable for administration to a human subject, and will have the desired immunogenic properties in combination with acceptable safety and reactogenicity. Typically, the formulated immunogenic composition is formulated in a single dose amount of at least 0.05 ml and no more than 2 ml, such as between 0.5 and 1.5 ml. For example, a single dose can be in the amount of between 0.5 and 0.5 ml, or between 0.1 and 2 ml, or between 0.5 and 1.5 ml, such as in the amount of 0.05 ml, 0.06 ml, 0.07 ml, 0.075 ml, 0.08 ml, 0.09 ml, 0.1 ml., 0.2 ml, 0.25 ml, 0.3 ml, 0.33 ml, 0.4 ml, 0.5 ml, 0.6 ml, 0.66 ml, 0.7 ml, 0.75 ml, 0.8 ml, 0.9 ml, 1.0 ml, 1.25 ml, 1.33 ml, 1.5 ml or 2 ml, or any intervening volume.

Although the composition can be administered by a variety of different routes, most commonly, the immunogenic compositions are delivered by an intramuscular, subcutaneous or intradermal route of administration. Generally, the vaccine may be administered subcutaneously, intradermally, or intramuscularly in a dose effective for the production of neutralizing antibody and protection. The vaccines are administered in a manner compatible with the dosage formulation, and in such amount as will be prophylactically and/or therapeutically effective. The quantity to be administered, which is generally in the range of 0.05-100 μg of each strain of inactivated virus per dose, depends on the subject to be treated, capacity of the subject's immune system to synthesize antibodies, and the degree of protection desired. Precise amounts of the vaccine to be administered may depend on the judgment of the practitioner and may be peculiar to each subject.

The vaccine may be given in a single dose schedule, or preferably a multiple dose schedule in which a primary course of vaccination may be with 1-10 separate doses, followed by other doses given at subsequent time intervals required to maintain and or reinforce the immune response, for example, at 1-4 months for a second dose, and if needed, a subsequent dose(s) after several months or years. The dosage regimen will also, at least in part, be determined by the need of the individual and be dependent upon the judgment of the practitioner. Examples of suitable immunization schedules include: a first dose, followed by a second dose between 7 days and 6 months, and an optional third dose between 1 month and two years post initial immunization, or other schedules sufficient to elicit titers of virus-neutralizing antibodies expected to confer protective immunity, for example selected to correspond to an established pediatric vaccine schedule. The generation of protective immunity against Dengue with an inactivated virus vaccine may reasonably be expected after a primary course of immunization consisting of 1 to 3 inoculations. These could be supplemented by boosters at intervals (e.g., every two years) designed to maintain a satisfactory level of protective immunity.

The following examples are provided to illustrate certain particular features and/or embodiments. These examples should not be construed to limit the invention to the particular features or embodiments described. It will be appreciated by those of skill in th art that the amounts, e.g., volumes, are provided as examples only, and that the scale can be modified (either increased or decreased) at the option of the practitioner. Similarly, the components used in purification, e.g., filters, columns, are not intended to be in any way limiting or exclusionary, and can be substituted for other components to achieve the same purpose at the discretion of the practitioner.

### EXAMPLES

### Example 1

### Purification Process for Producing Purified Inactivated Dengue Virus

Dengue virus is grown in Vero cells and purified essentially as described in WO 2010/094663. For example, Dengue virus is grown in Vero cells, e.g., in animal free medium. Typically, the cells are maintained in a stationary preculture phase (e.g., in T flasks or a cell factory), in an animal free (AF) medium, such as the commercially available VPSFM medium from Invitrogen. The cells are then expanded in a bioreactor, typically attached to microcarriers (such as Cytodex 1), and fed by either perfusion or batch modes. Once the cells have reached a suitable density, the cells are infected at suitable MOI (e.g., 0.01-0.1, for example 0.05) with virus, either in serum-containing (e.g., 1.5%) or AF medium. When employing serum-containing medium, after an initial infection phase (typically of approximately 2 days), the medium is typically exchanged to AF medium. Optionally, the AF medium is initially or periodically supplemented with glucose, amino acids or the like.

After a suitable period for viral growth, e.g., between a minimum of 6 and 8 days, virus is harvested from the cells. Optionally, virus can be harvested incrementally at intervals (for example intervals of 2 days) starting at approximately 6 days post-infection.

An exemplary purification process is illustrated schematically in FIG. 2A. A modified purification process is illustrated schematically in FIG. 2B. Although essentially similar to the process of FIG. 2A, the process includes the following modifications. Following inactivation with formaldehyde, the step of neutralizing free formalin in the bulk by addition of sodium bisulfite is eliminated. Elimination of sodium bisulfite neutralization significantly increases the yield in subsequent filtration steps. Free formalin is eliminated by a diafiltration step.

An alternative purification process is illustrated in FIGS. 2C and D. Following harvest, the medium containing the virus is clarified, typically through a series of decreasing pore sizes (e.g., 8 g, 0.6 g, 0.45 g, 0.2 g). The virus suspension is then concentrated and the medium exchanged for buffer, e.g., by ultrafiltration and diafiltration, followed by further filtration and size exclusion chromatography (SEC), using, e.g., Sephacryl s-400HRor Sepharose 4 FF resins. Optionally, prior to further processing, the clarified virus suspension is inactivated by exposure to UV irradiation (between 100-500, e.g., 200 J/m²), either before or after the concentration step. Optionally, the size exclusion chromatography step can be followed by one or more steps, e.g., Sartobind-Q membrane chromatography (in negative mode) and filtration to remove residual DNA. It is generally preferred that residual DNA be reduced to less than or equal to 100 pg DNA per μg protein (or to less than 100 pg/dose).

The virus is then inactivated, by exposure to formaldehyde (at approximately 100 μg/ml) for a period typically between 7 and 10 days at room temperature. Optionally, the suspension is filtered (e.g., 0.220 at an intermediate time point, such as at day 2, 3, 4, or 5, to remove aggregates and improve formaldehyde exposure. Following inactivation, a poloxamer surfactant can be added to the buffer, prior to ultrafiltration/diafiltration to remove the formaldehyde and place the purified inactivated Dengue virus in a buffer suitable for storage. The purified inactivated Dengue virus is then finally sterile filtered prior to storage as a bulk preparation of inactivated Dengue virus. Optionally, sucrose is added to the final formulation of the bulk preparation.

### Example 2

### Formulation of Exemplary Immunogenic Compositions

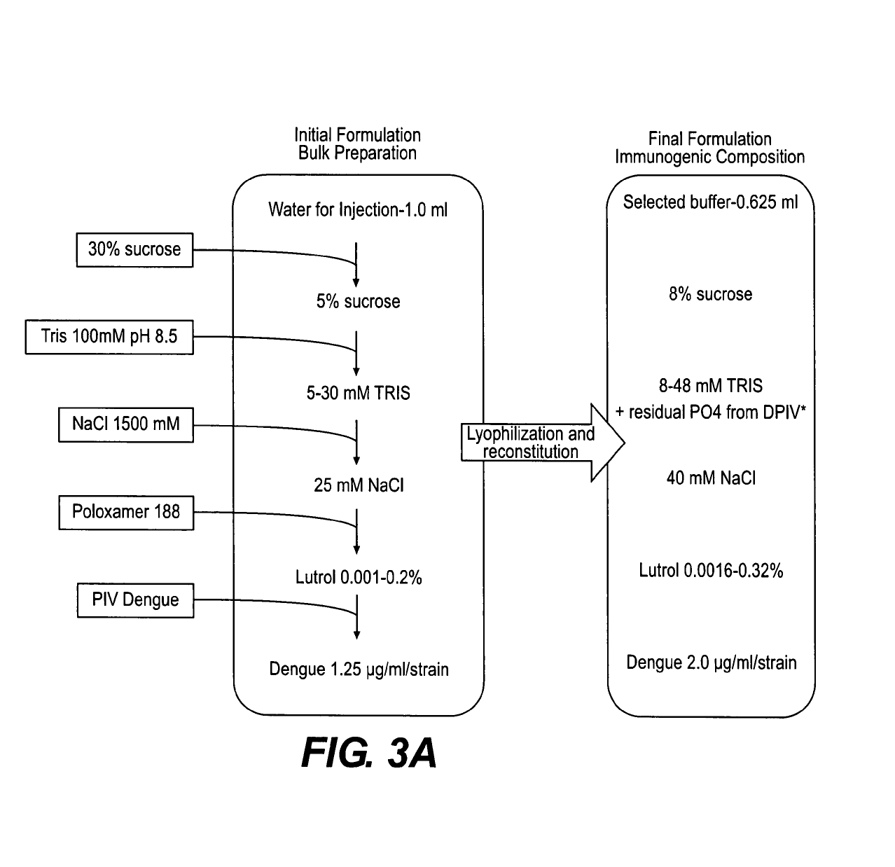

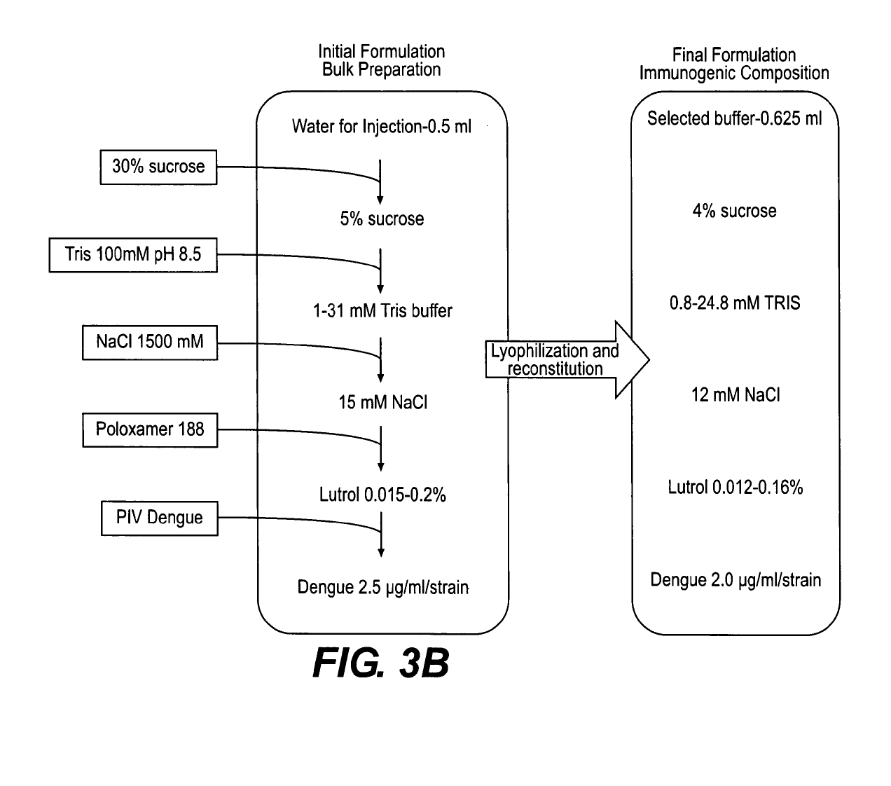

Formulation of purified inactivated Dengue virus was assessed under different conditions to solve the problem of loss of product during storage, lyophilization and subsequent handling. The following variables were evaluated in the formulation of the bulk preparation of inactivated Dengue virus (3.3 μg/ml per strain): Phosphate buffer (pH 8.5) concentration (5, 15, 30 mM), poloxamer surfactantconcentration (0, 0.001%, 0.2%), in the presence of 5% sucrose, 25 mM NaCl in water for injection. The bulk was lyophilized and reconstituted in 0.625 ml of resuspension solution. The formulation process is illustrated schematically in FIG. 3A. Alternative formulations, replacing phosphate buffer with Tris buffer and reducing the unit volume to generate a single dose from 1.0 to 0.5 ml prior to lyophilization is illustrated in FIG. 3B. It will be appreciated that the volume adjustments and buffer modifications are independent variables and either or both modifications can be made separately or in tandem.

Stability of the dried cakes was assessed following incubation for 7 days at 4° C. and 37° C. Cake aspect and residual humidity of the dried product were evaluated. The lyophilized cakes were then reconstituted in NaCl, or in different buffers to assess pH stability. A pre-lyophilization volume of 1.5 ml was used resulting in a 2.4 fold concentration factor after reconstitution with 0.625 ml of resuspension solution. The resulting resuspended immunogenic compositions were analyzed for cake quality and by intrinsic fluorescence 280/320 nm, nitrogen content, Elisa, Dynamic Light Scattering, Nephelometry, pH, and osmolality.

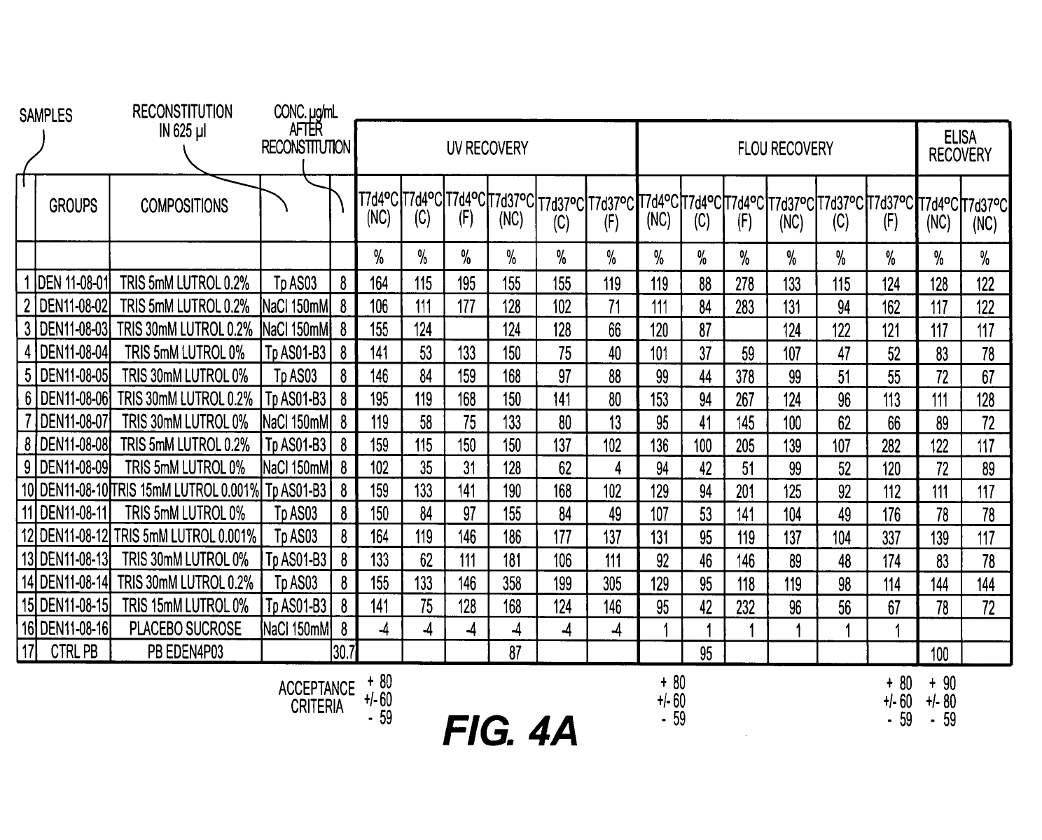

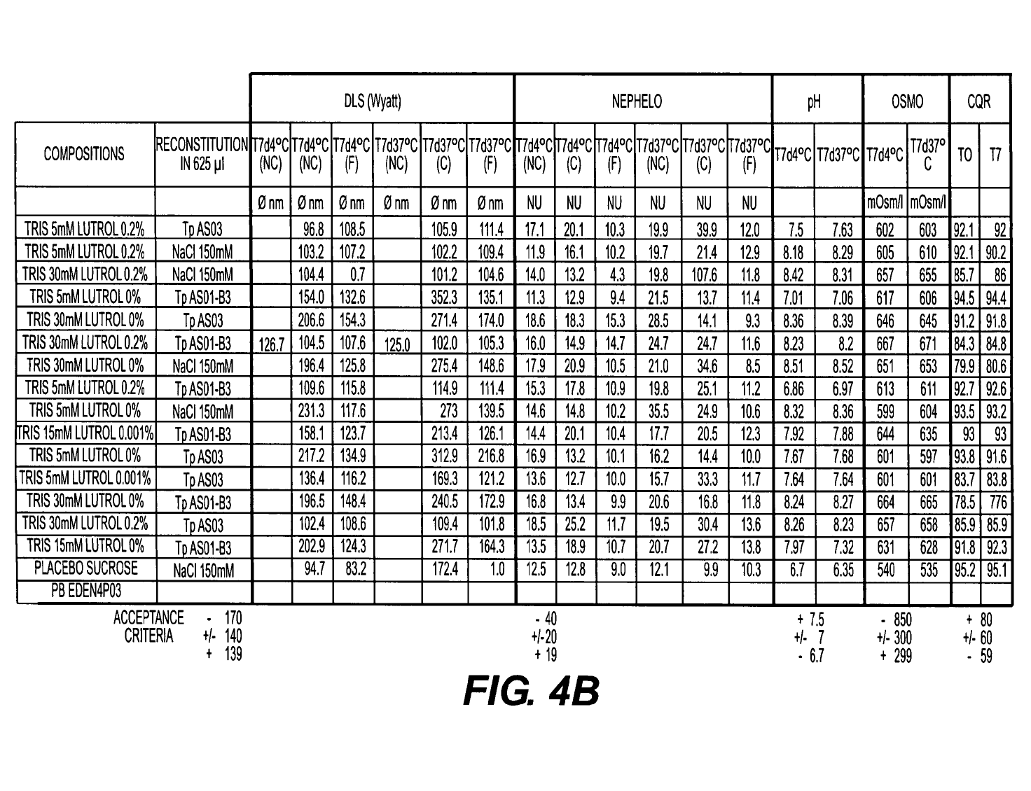

Exemplary results are shown in FIGS. 4A-B.

These results demonstrated that at concentrations from 0.001% to 0.2% surfactant (Lutrol™) yielded a full recovery of protein in all tested resuspension solutions. This contrasted with the loss of protein content in the absence of surfactant. Without being bound by theory, it is believed that the loss of protein in the absence of surfactant was due aspecific adsorption on vials, which is prevented by the addition of a suitable surfactant. At a concentration of 0.2%, the surfactant also prevented aggregation of viral particles.

The buffer concentration and composition of the resuspension solution had no impact on initial product recovery.

Immunological assessment by ELISA demonstrated that immunological epitopes of the inactivated Dengue virus were preserved following lyophilization and reconstitution. Further details concerning lyophilization and reconstitution are provided in Example 3.

### Example 3

### Lyophilization Bulk Preparation and Reconstitution into an Immunogenic Composition

Purified inactivated Dengue virus was formulated into a bulk preparation as described above and shown in FIG. 3B, to the following specification: 5% sucrose, 1-31 mM Tris buffer, 15 mM NaCl, 0.015-0.2% Poloxamer 188 with purified inactivated Dengue virus at 1.25 μg/strain for each of the four strains. For lyophilization, the bulk preparation was distributed into 0.5 ml aliquots. The bulk preparation was then lyophilized in the following 74 hour Freeze Drying Cycle: Freezing to <−52° C. over 1 hour at 1 atmosphere (Atm.); Primary drying at 45 μbar as follows: 1) Cooling from −52° C. to −32° C. over 3 hours; 2) 32° C. for 32 hours; 3) sequential decline in temperature in 1° C. increments with a 10 minute decrease followed by a 2 hour 25 minute maintenance period (total 7 hour 55 minutes); 4)-28° C. for 9 hours; Secondary Drying as follows:

Temparature increase from −28° C. to 37° C. over 9 hours at 45 μbar followed by 37° C. for 12 hours at 27 μbar. The lyophilized samples were then equilibrated to between 2 and 8° C. to complete the cycle. The resulting lyophilized product (“cake”) was incubated for 24 hours at room temperature after rehydration or 1 month at 37° C. or 3 months at −20° C., or 5 months at 4° C. in lyophilized form to assess stability.

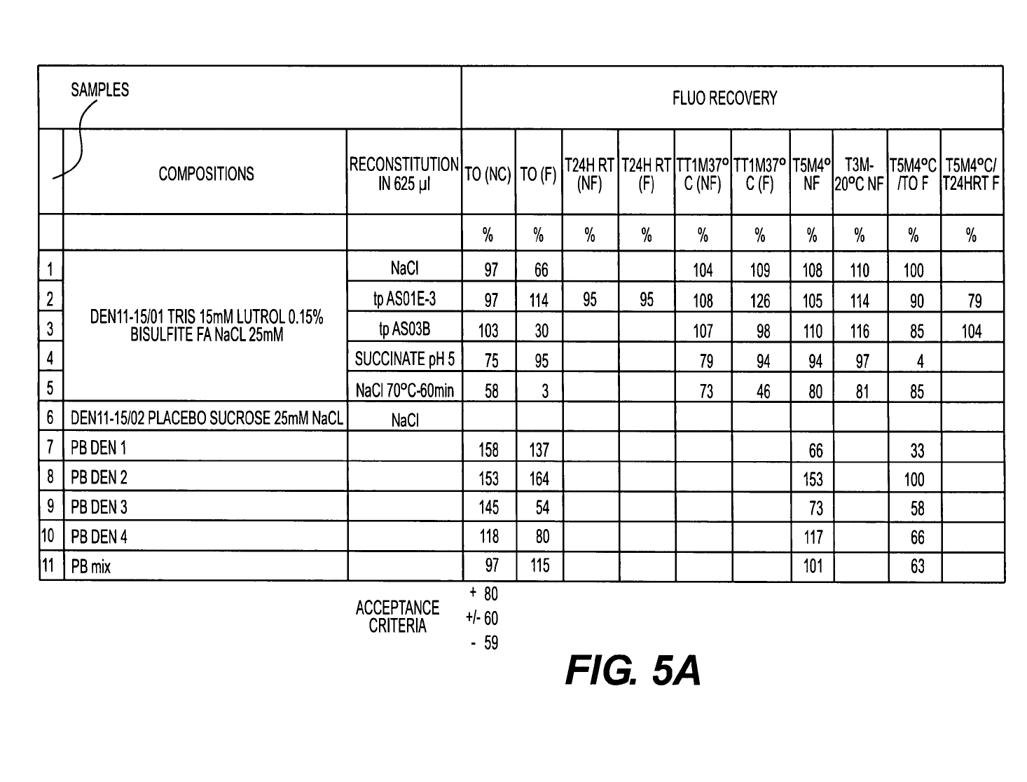

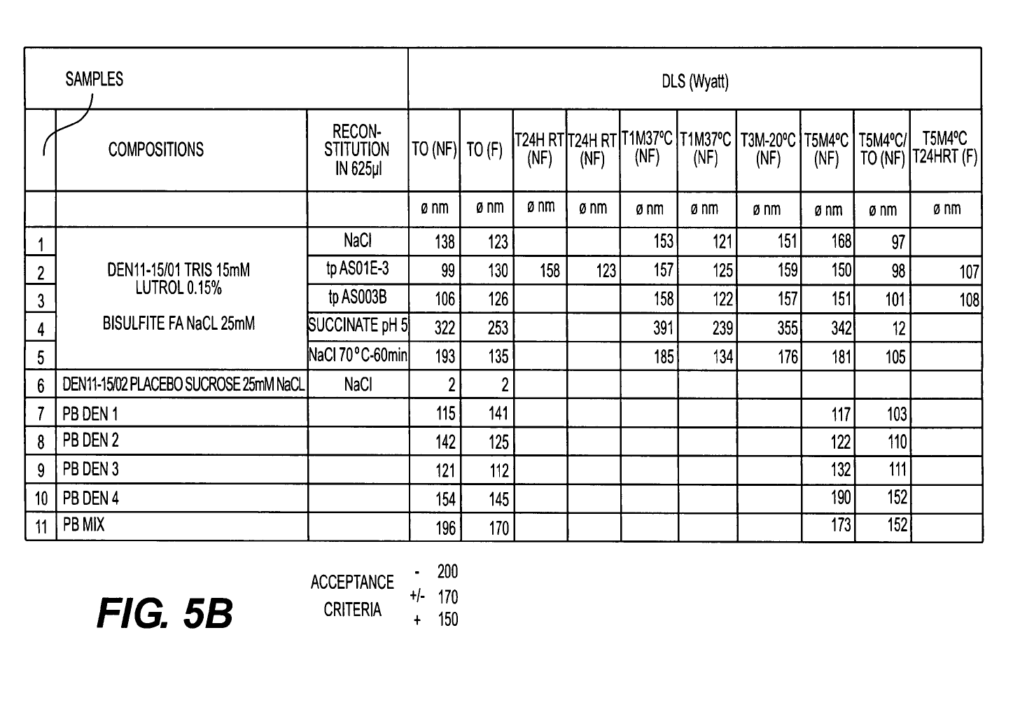

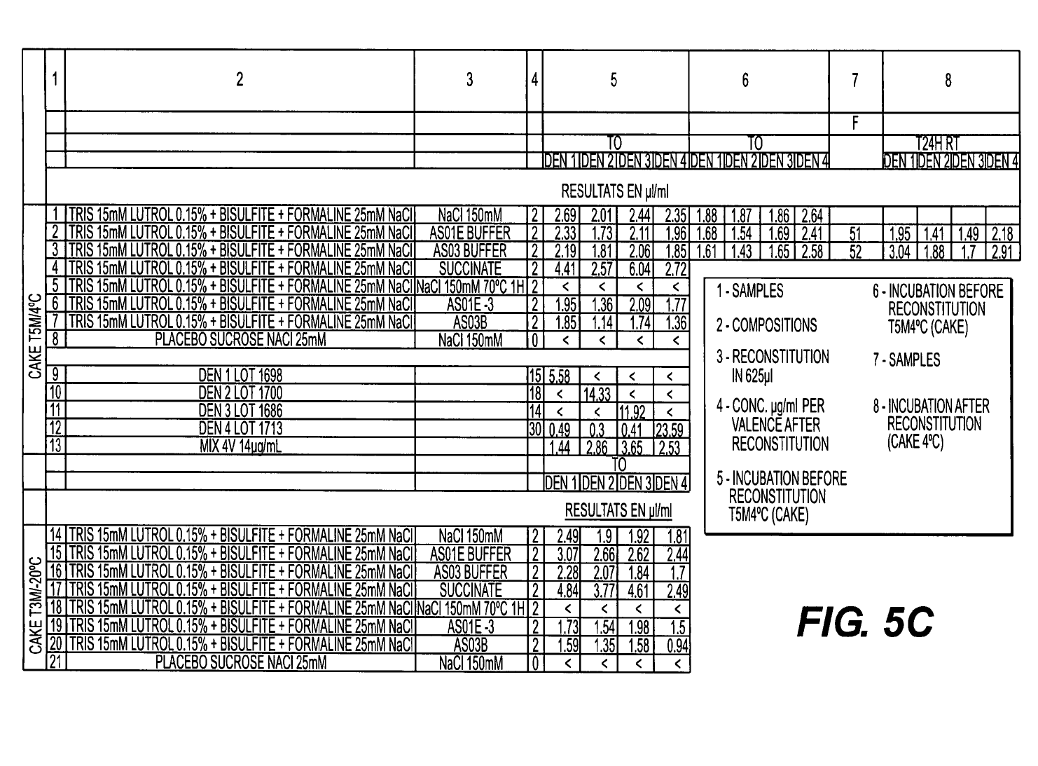

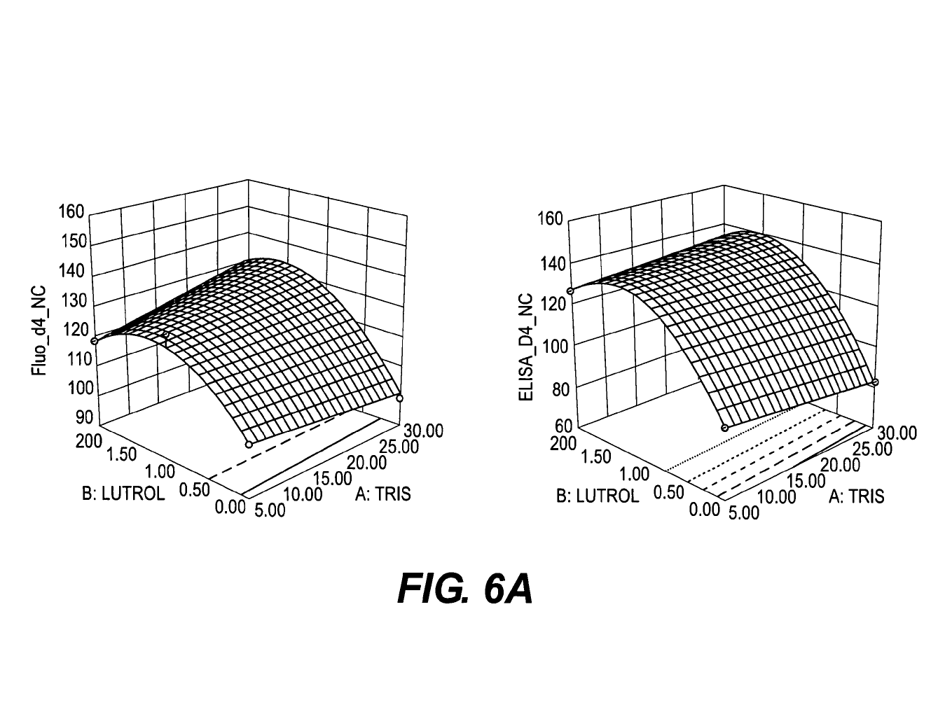

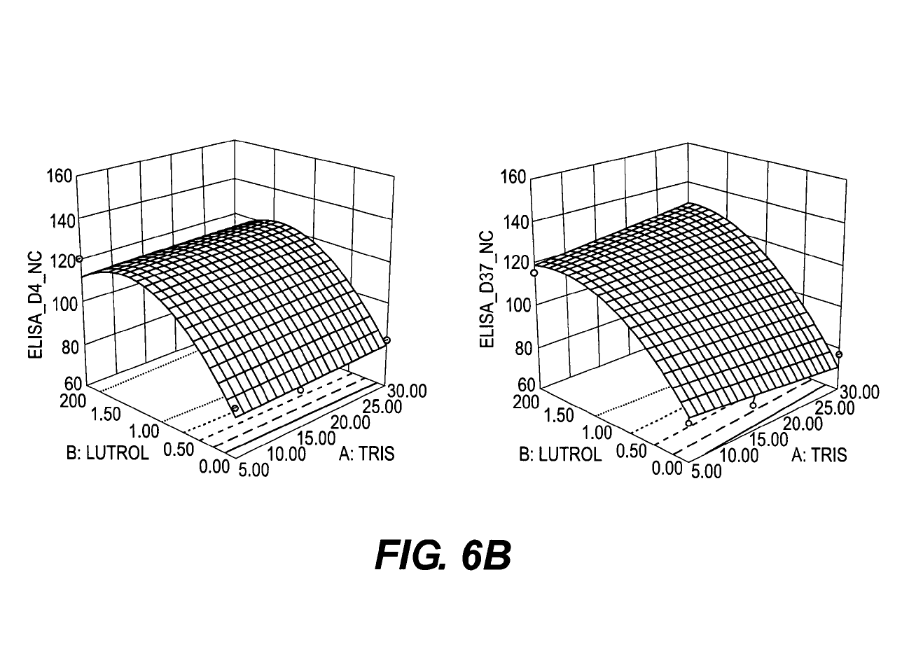

Upon reconstitution into 0.625 ml in the selected buffer, the resulting concentration in the immunogenic composition was as follows: 4% sucrose, 0.8-24.8 mM phosphate, 12 mM NaCl, 0.012-0.16% Poloxamer 188, purified inactivated Dengue virus at 2.0 μg. The resulting immunogenic compositions were assayed for quality, stability and immunogenicity by the following assessments: intrinsic fluorescence, DLS, Nephelometry, pH, osmolality and ELISA. Representative results are shown in FIGS. 5A-C. FIGS. 6A and 6B graphically illustrate stability characteristics (intrinsic fluorescence and ELISA, respectively) upon reconstitution in buffer of lyophilized preparations in the presence and absence of surfactant. All data were in expected values, with a clear increase in recovery for the surfactant-containing formulations as compared to surfactant-free formulations. No impact of buffer (Tris) concentration was observed over the ranges tested. Similar results were obtained in a variety of reconstitution buffers (e.g., to obtain liquid immunogenic compositions suitable for administration with different adjuvants). These results demonstrated that lyophilization and reconstitution in the presence of Poloxamer surfactant and buffer to maintain pH at or above neutral resulted in favorable stability and immunogenicity in a variety of buffer compositions.

1-119. (canceled)

120. A bulk preparation of inactivated Dengue virus or an immunogenic composition comprising:
one or more purified inactivated Dengue viruses;
a buffering agent; and
a poloxamer surfactant.

121. The immunogenic composition of claim 120, further comprising an adjuvant.

122. The immunogenic composition of claim 121, wherein the adjuvant comprises an aluminum salt, optionally at least one of aluminum hydroxide and aluminum phosphate.

123. The immunogenic composition of claim 122, further comprising at least one additional immunostimulatory component, optionally wherein the at least one additional immunostimulatory component comprises one or more of an oil and water emulsion, a liposome, a lipopolysaccharide, a saponin, and an oligonucleotide.

124. The immunogenic composition of claim 121, wherein the adjuvant is an aluminum-free adjuvant, optionally comprising one or more immunostimulatory components selected from the group consisting of: an oil and water emulsion, a liposome, a lipopolysaccharide, a saponin and an oligonucleotide.

125. A bulk preparation of inactivated Dengue virus or an immunogenic composition comprising:
at least one purified inactivated Dengue viruses adsorbed onto an aluminum salt;
a buffering agent; and
a surfactant.

126. The immunogenic composition of claim 125, further comprising at least one additional immunostimulatory component, optionally which comprises one or more of an oil and water emulsion, a liposome, a lipopolysaccharide, a saponin, and an oligonucleotide.

127. The immunogenic composition of claim 126, wherein the one or more immunostimulatory component comprises 3-deacylated monophosphoryl lipd A (3D-MPL), QS21, a DNA oligonucleotide comprising at least one unmethylatated CpG and/or a liposome.

128. The bulk preparation of inactivated Dengue virus or immunogenic composition of claim 120, wherein the surfactant is suitable for intramuscular, subcutaneous, transcutaneous or intradermal administration.

129. The bulk preparation of inactivated Dengue virus or immunogenic composition of claim 120, wherein the surfactant is selected from the group consisting of a poloxamer, macrogol 15 hydroxy stearate, a polysorbate, an octoxinol, a polidocanol, a polyoxyl stearate, a polyoxyl castor oil, an N-octyl-glucoside, and combinations thereof.

130. The bulk preparation of inactivated Dengue virus or immunogenic composition of claim 120, wherein the poloxamer surfactant has a molecular weight of at least 4500 kD and/or no more than 15,000 kD.

131. The bulk preparation of inactivated Dengue virus or immunogenic composition of claim 120, wherein the surfactant is present in an amount of at least 0.001% (w/v) and/or no more than 1.0% (wt/v).

132. The bulk preparation of inactivated Dengue virus or immunogenic composition of any claim 120, wherein the immunogenic composition comprises a plurality of purified inactivated Dengue viruses of different serotypes, optionally comprising a plurality of viruses that elicit an immune response to each DEN-1, DEN-2, DEN-3 and DEN-4.

133. The bulk preparation of inactivated Dengue virus or immunogenic composition of claim 120, wherein at least one of the purified inactivated Dengue viruses is an attenuated Dengue virus, a recombinant Dengue virus or a chimeric virus comprising a first Dengue virus nucleic acid and a second flavivirus nucleic acid.

134. The bulk preparation of inactivated Dengue virus or immunogenic composition of claim 120, wherein the buffering agent comprises one or more of sodium phosphate, potassium phosphate and Tris(hydroxymethyl)aminomethane.

135. The bulk preparation of inactivated Dengue virus or immunogenic composition of claim 120, wherein the buffering agent maintains the pH in a liquid composition at or above pH 6.4.

136. The bulk preparation of inactivated Dengue virus or immunogenic composition of claim 120, further comprising at least one of a glass forming sugar and a glass forming polyol, optionally wherein the glass forming sugar or polyol is selected from the group consisting of: sucrose, trehalose, mannose, mannitol, raffinose, lactitol, sorbitol and lactobionic acid, glucose, maltulose, iso-maltulose, lactulose, maltose, lactose, iso-maltose, maltitol, palatinit, stachyose, melezitose, dextran or a combination thereof.

137. The immunogenic composition of claim 120, wherein the immunogenic composition is formulated for administration to a human subject.

138. The bulk preparation of inactivated Dengue virus or immunogenic composition of claim 120, wherein the one or more purified inactivated Dengue virus is inactivated by at least one of a chemical inactivating agent, a physical inactivating agent and an irradiating inactivating agent, optionally by exposure to at least one of formaldehyde, betapropiolactone (BPL), hydrogen peroxide, ultraviolet irradiation and gamma irradiation.

139. A lyophilized preparation of inactivated Dengue virus comprising:
at least one purified inactivated Dengue virus;
and
a poloxamer surfactant.

140. A method of formulating a bulk preparation of inactivated Dengue virus or an immunogenic composition comprising
providing a solution comprising a buffering agent and a surfactant; and
admixing with the solution one or more purified inactivated Dengue viruses.
